(19) United States
(12) Patent Application Publication
(10) Pub. No.: US 2026/0216300 A1
(43) Pub. Date: Jul. 30, 2026

(54) POLYMERIZED HEMOGLOBIN SIZE FRACTIONATED VIA TANGENTIAL FLOW FILTRATION

(71) Applicant: Ohio State Innovation Foundation, Columbus, OH (US)

(72) Inventor: Andre PALMER, Westerville, OH (US)

(21) Appl. No.: 19/147,394

(22) PCT Filed: Jan. 12, 2024
(86) PCT No.: PCT/US24/11474

§ 371 Date: Jul. 11, 2025

Related U.S. Application Data
(60) Provisional application No. 63/479,865, filed on Jan. 13, 2023.

(51) Int. Cl. A61K 38/42; A61K 9/00; A61K 31/195; A61K 31/573; A61K 38/38; A61K 45/06; A61K 47/02; A61K 47/10; A61K 47/12; A61K 47/18; B01D 61/14

(52) U.S. Cl. CPC A61K 38/42; A61K 9/0026; A61K 31/195; A61K 31/573; A61K 38/385; A61K 45/06; A61K 47/02; A61K 47/10; A61K 47/12; A61K 47/183; B01D 61/146; B01D 2315/10

(57) ABSTRACT

Described herein hemoglobin-based oxygen carrier (HBOC) composition comprising a polymerized hemoglobin (Poly-Hb). The PolyHb can be purified using sequential tangential flow filtration processes to create fractions which contain minimal quantities of low molecular weight and high molecular weight hemoglobin species.

### CROSS REFERENCE TO RELATED APPLICATIONS

This application claims the benefit of priority to U.S. Provisional Application No. 63/479,865, filed Jan. 13, 2023, which is incorporated by reference herein in its entirety.

### STATEMENT REGARDING FEDERALLY SPONSORED RESEARCH OR DEVELOPMENT

This invention was made with government support under Grant Nos. R01HL156526, R01HL158076, R01HL159862, and R01HL162120 awarded by the National Institutes of Health. The government has certain rights in the invention.

### BACKGROUND

There is a continuing need for a blood-substitute to treat or prevent hypoxia resulting from blood loss (e.g., from acute hemorrhage or during surgical operations), resulting from anemia (e.g., pernicious anemia or sickle cell anemia), or resulting from shock (e.g., volume deficiency shock). The use of blood and blood fractions as in these capacities as a blood-substitute is fraught with disadvantages. For example, the use of whole blood often is accompanied by the risk of transmission of hepatitis-producing viruses and AIDS-producing viruses which can complicate patient recovery or result in patient fatalities. Additionally, the use of whole blood requires blood-typing and cross-matching to avoid immunohematological problems and inter-donor incompatibility. Further whole blood must be refrigerated and expires, complicating storage and provision of whole blood in many situations.

Human hemoglobin, as a blood-substitute, possesses the ability to transport and deliver oxygen, but it has the disadvantage of rapid elimination from circulation by the renal route and through vascular walls, resulting in a very short, and therefore, a typically unsatisfactory half-life. In addition, extravasation of hemoglobin into the tissue space leads to iron deposition which results in oxidative tissue injury.

Non-human hemoglobin suffers from the same deficiencies as human hemoglobin.

Previously, at least four other types of blood-substitutes have been evaluated, including perfluorochemicals, synthesized hemoglobin analogues, liposome-encapsulated hemoglobin, and chemically-modified hemoglobin. However, many of these blood-substitutes have typically had short intravascular retention times, being removed by the circulatory system as foreign substances or lodging in the liver, spleen, and other tissues.

Thus, in spite of the recent advances in the preparation of hemoglobin-based blood-substitutes, the need has continued to exist for a blood-substitute which has levels of contaminants, such as endotoxins, bacteria, viruses, phospholipids and non-hemoglobin proteins, which are sufficiently low to generally prevent an immune system response and any toxicological effects resulting from an infusion of the blood-substitute. In addition, the blood-substitute must also be capable of transporting and transferring adequate amounts of oxygen to tissue under ambient conditions and must have a good intravascular retention time.

Further, it is preferred that the blood-substitute 1) can be transfused to most recipients without cross-matching or sensitivity testing, and 2) can be stored with minimum amounts of refrigeration for long periods.

### SUMMARY

In accordance with the purposes of the disclosed materials and methods, as embodied and broadly described herein, the disclosed subject matter, in one aspect, relates to a hemoglobin-based oxygen carrier (HBOC) composition comprising a polymerized hemoglobin (PolyHb), wherein the composition comprises less than 10% by weight hemoglobin species having a molecular weight of less than 500 kDa, based on the total weight of the HBOC composition; and wherein the composition comprises less than 10% by weight hemoglobin species having a molecular weight of greater than 750 kDa, based on the total weight of the HBOC composition.

In another aspect, provided is a hemoglobin-based oxygen carrier (HBOC) composition comprising a polymerized hemoglobin (PolyHb), wherein the composition comprises less than 10% by weight hemoglobin species having a molecular weight of less than 500 kDa, based on the total weight of the HBOC composition; and wherein the composition comprises less than 10% by weight hemoglobin species having a hydrodynamic diameter of greater than 50 nm, based on the total weight of the HBOC composition.

In another aspect, provided is a hemoglobin-based oxygen carrier (HBOC) composition comprising a polymerized hemoglobin (PolyHb), wherein the composition comprises less than 10% by weight hemoglobin species having a molecular weight of less than 500 kDa, based on the total weight of the HBOC composition; and wherein the composition comprises less than 10% by weight hemoglobin species having a hydrodynamic diameter of greater than 0.1 microns, based on the total weight of the HBOC composition.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate certain examples of the present disclosure and together with the description, serve to explain, without limitation, the principles of the disclosure. The application includes reference to the accompanying figures, in which:

FIG. 1 is a schematic illustration of the PolyhHb reaction system and reaction timeline. The schematic shows the 2 L benchtop scale polymerization reactor used to synthesize T-state PolyhHb, and the associated reaction timeline. (PolyhHb, polymerized human hemoglobin)

FIG. 2 is a schematic illustration of the PolyhHb TFF purification system. The figure shows the TFF modules used to purify each of the four PolyhHb MW brackets. Stage 1 represents the clarification step, and TFF modules with MW cut offs (MWCOs) of 300 kDa, 500 kDa, 750 kDa, and 0.2 μm were used to prepare PolyhHb-B1 to PolyhHb-B4, respectively. Stage 2 represents the purification and concentration step, and TFF modules with MWCOs of 50 kDa, 100 kDa, 500 kDa, and 750 kDa were used to prepare PolyhHb-B1 to PolyhHb-B4, respectively.

FIGS. 3A-3C show the SEC-HPLC analysis and hydrodynamic diameter of PolyhHb and hHb. FIG. 3A shows the normalized intensity distribution of the elution time for hHb, PolyhHb-B1, PolyhHb-B2, PolyhHb-B3, and PolyhHb-B4 measured using SEC-HPLC. Distributions were taken from the 413 nm absorbance normalized against the maximum intensity. FIG. 3B shows the effective hydrodynamic diameter of hHb, PolyhHb-B1, PolyhHb-B2, PolyhHb-B3, and PolyhHb-B4 were measured using DLS. FIG. 3C shows denaturing SDS-PAGE of hHb, PolyhHb-B1, PolyhHb-B2, PolyhHb-B3, and PolyhHb-B4.

FIG. 4A shows the O₂ equilibrium curve (OEC) and FIG. 4B shows the O₂ offloading kinetics for PolyhHb-B1, PolyhHb-B2, PolyhHb-B3, PolyhHb-B4 and hHb. Lines represent the mean from all batches. Shaded areas indicate the standard error for each PolyhHb bracket. FIG. 4C shows the pseudo-first-order Hp binding kinetics of PolyhHb/hHb. The normalized fluorescence changes (λ_{excitation}=285 nm, λₑₘᵢₛₛᵢₒₙ=310 nm) were fit to a monoexponential equation. FIG. 4D shows the second order Hp binding kinetics of PolyhHb-B1, PolyhHb-B2, PolyhHb-B3, PolyhHb-B4, and hHb. The second order Hp binding rate constant was obtained via performing a linear fit of the pseudo-first order Hp binding rate constant as a function of the hHb concentration.

FIG. 5 is a plot showing the auto-oxidation kinetics of PolyhHb and hHb. Auto-oxidation of 0.775 mM (heme basis) PolyhHb and hHb was measured via UV-visible spectrometry over 24 hours at 37° C. Biphasic auto-oxidation kinetics were observed for PolyhHb and were fit to two line segments. The first order auto-oxidation rate constants were denoted as k_{ox,fast} and k_{ox,slow}, respectively. Monophasic auto-oxidation kinetics were observed for hHb and fit to a single rate constant.

FIGS. 6A-6F show the pharmacokinetic parameter estimates of PolyhHb brackets. PK parameter estimates were made from plasma concentrations obtained over the 48 hours following a 25% ET. Plasma samples were measured using UV-visible spectrometry and the plasma versus time data for each dosed animal was analyzed using a non-compartmental analysis with Phoenix 64 WinNonlin software. FIG. 6A shows the plasma concentration versus time curves for each PolyhHb bracket, dash lines PolyhHb-B1 and -B2 are extrapolated to zero. FIG. 6B demonstrates that the concentration of PolyhHb in plasma after 25% ET was not different across groups and FIG. 6C shows the circulating half-life of brackets derived from the end of ET until Cₗₐₛₜ (48 hours). FIGS. 6D and 6E demonstrate that the exposure parameters, AUC_{0-∞} (●) and total clearance (▴) increased from PolyhHb-B1 to PolyhHb-B3 and reached saturation following ET with PolyhHb-B4. FIG. 6F summarizes the observation of MW dependent exposure parameter saturation. All data are represented as individual values with Mean±SD and statistical significance represented as: * p<0.05, **p<0.01, ***p<0.001, ****p<0.0001.

FIGS. 7A-7D show the tissue distribution of PolyhHb brackets by primary clearance organs. Sections were prepared from tissue collected following 90% clearance of 25% ET with PolyhHb bracketed materials. As shown in FIG. 7A, kidney tissue from top to bottom shows a visual representation of iron distribution in non-treated (NT) and PolyhHb-B1, -B2, -B3 and -B4 ET guinea pig kidney sections. PolyhHb-B1 and -B2 show diffuse iron staining in renal cortical proximal and distal tubule regions at 4×, with boxed area shown at 20×. As shown in FIG. 7B, liver tissue from top to bottom shows a visual representation of iron distribution in PolyhHb ET guinea pig liver sections indicating minimal hepatic clearance after ET with PolyhHb-B1. Scale bars indicate 30 microns (μm). FIGS. 7C and 7D show the kidney and liver image quantitative group comparisons. All data are represented as Mean±SD and statistical significance represented as: *p<0.05, **p<0.01, ***p<0.001, ****p<0.0001.

FIGS. 8A-8C show the tissue metabolism of PolyhHb brackets by primary clearance organs. Sections were prepared from tissue collected following 90% clearance of 25% ET with PolyhHb bracketed materials. As shown in FIG. 8A, left panel, kidney tissue from top to bottom shows a visual representation of HO-1 distribution in non-treated (NT) and PolyhHb-B1, -B2, -B3 and -B4 ET guinea pig kidney sections. PolyhHb-B1 and -B2 show HO-1 expression in renal cortical proximal and distal tubule regions (indicated by arrows) surrounding glomeruli at 60× magnification. As shown in FIG. 8B, right panel, liver tissue from top to bottom shows a visual representation of HO-1 expression in hepatic Kupffer cells indicated by arrows in PolyhHb-B2, -B3 and -B4. Scale bars represent 60 microns (μm). FIGS. 8B and 8C show the kidney and liver image quantitative group comparisons. All data are represented as Mean±SD and statistical significance is represented as: *p<0.05, **p<0.01, ***p<0.001, ****p<0.0001.

FIGS. 9A-9C show the hemodynamics of exchange transfusion with PolyhHb. FIG. 9A is a plot showing the mean arterial pressure (MAP) in response to exchange transfusion with HBOCs of varied sizes over the 20-minute baseline period and 90-minute observation period. FIG. 9B is a plot comparting the area under the MAP curve (AUC) for each HBOC. FIG. 9C is a plot showing the pulse pressure in response to exchange transfusion with HBOCs of varied sizes over the 20-minute baseline period and 90 minute observation period. *p<0.05, **p<0.01, ***p<0.001.

FIG. 10 shows representative electrocardiogram (ECG) traces. Normal guinea pig ECG in sinus rhythm is shown followed by normal cardiac rhythms during 25% ET with PolyhHb-B1, PolyhHb-B2 and PolyhHb-B3. No periods of abnormal rhythm or rate were observed following transfusion with PolyhHb-B1, -B2 or -B3. However, runs of aberrant cardiac rhythms during 25% ET with PolyhHb-B4 were observed as T-wave inversion, shown as red boxes throughout the ECG interval. This observation is indicative of cardiac ischemia and indicates a relevant preclinical safety pharmacology adverse effect.

Reference will now be made in detail to specific aspects of the disclosed materials, compounds, compositions, formulations, articles, and methods, examples of which are illustrated in the accompanying Examples and Figures.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### General Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a compound”, “a composition”, or “a disorder”, includes, but is not limited to, two or more such compounds, compositions, or disorders, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “tangential-flow filtration” refers to a process in which the fluid mixture containing the components to be separated by filtration is recirculated at high velocities tangential to the plane of the filtration membrane to reduce fouling of the filter. In such filtrations a pressure differential is applied along the length of the filtration membrane to cause the fluid and filterable solutes to flow through the membrane (i.e., filter). This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is processed (e.g., continually processed) downstream.

As used herein, the term “ultrafiltration” is used for processes employing membranes rated for retaining solutes having a molecular weight between about 1 kDa and 1000 kDa.

As used herein, the term “reverse osmosis” refers to processes employing membranes capable of retaining solutes of a molecular weight less than 1 kDa such as salts and other low molecular weight solutes.

As used herein, the term “microfiltration” refers to processes employing membranes in the 0.1 to 10 micron pore size range.

As used herein, the expression “transmembrane pressure” or “TMP” refers to the pressure differential gradient that is applied along the length of a filtration membrane to cause fluid and filterable solutes to flow through the filter.

As used herein, “glutaraldehyde” refers to C₅H₈O₂ or OCH(CH₂)₃CHO, is a transparent oily, liquid with a pungent odor. It is a dialdehyde comprised of pentane with aldehyde functions at C-1 and C-5. Alternatives to glutaraldehyde can include carboiimide, diisocyanates and polyepoxy compounds, as well as Genipin (Challenge Bioproducts Co., Ltd., Taiwan), epigallocatechin gallate (Sigma, St. Louis, MO), and grape seed proanthocyanidin (PureBulk, Inc., Roseburg, OR).

### HBOC Compositions

The present disclosure provides for a hemoglobin-based oxygen carrier (HBOC) composition. The HBOC composition can comprise a polymerized hemoglobin (PolyHb). The PolyHb can be purified using sequential filtration processes (e.g., sequential tangential flow filtration) to create fractions which contain minimal quantities of low molecular weight and high molecular weight hemoglobin species.

For example, in some embodiments, the composition can comprise less than 10% by weight hemoglobin species having a molecular weight of less than 500 kDa, based on the total weight of the HBOC composition, and less than 10% by weight hemoglobin species having a molecular weight of greater than 750 kDa, based on the total weight of the HBOC composition.

In some embodiments, the composition can comprise less than 10% by weight hemoglobin species having a molecular weight of less than 500 kDa, based on the total weight of the HBOC composition; and less than 10% by weight hemoglobin species having a hydrodynamic diameter of greater than 50 nm, based on the total weight of the HBOC composition.

In some embodiments, the composition can comprise less than 10% by weight hemoglobin species having a molecular weight of less than 500 kDa, based on the total weight of the HBOC composition; and less than 10% by weight hemoglobin species having a hydrodynamic diameter of greater than 0.1 microns, based on the total weight of the HBOC composition.

As used herein, “polymerized hemoglobin”, also referred to as “polymerized Hb” or “PolyhHb”, refers to a class of hemoglobin (Hb) based O₂ carrier (HBOC) that can be synthesized and purified at large scale such that it can transport and offload O₂ to support cellular metabolism, while not demonstrating cytotoxic side-effects. In some embodiments, hemoglobin is polymerized with glutaraldehyde. As used herein, “hemoglobin species, or “hemoglobin (Hb)”, refers to the protein inside red blood cells that carries oxygen from the lungs to tissues and organs in the body and carries carbon dioxide back to the lungs. It can include four protein chains, two alpha chains and two beta chains, each with a ring-like heme group containing an iron atom. Oxygen can bind reversibly to these iron atoms and can be transported through blood.

In some embodiments, the composition can comprise less than 5% by weight hemoglobin species having a molecular weight of less than 500 kDa, based on the total weight of the HBOC composition, such as less than 2.5% by weight hemoglobin species having a molecular weight of less than 500 kDa, less than 1% by weight hemoglobin species having a molecular weight of less than 500 kDa, less than 0.5% by weight hemoglobin species having a molecular weight of less than 500 kDa, less than 0.1% by weight hemoglobin species having a molecular weight of less than 500 kDa, or less than 0.05% by weight hemoglobin species having a molecular weight of less than 500 kDa.

In some embodiments, the composition can comprise less than 5% by weight hemoglobin species having a molecular weight of less than 300 kDa, based on the total weight of the HBOC composition, such as less than 2.5% by weight hemoglobin species having a molecular weight of less than 300 kDa, less than 1% by weight hemoglobin species having a molecular weight of less than 300 kDa, less than 0.5% by weight hemoglobin species having a molecular weight of less than 300 kDa, less than 0.1% by weight hemoglobin species having a molecular weight of less than 300 kDa, or less than 0.05% by weight hemoglobin species having a molecular weight of less than 300 kDa.

In some embodiments, the composition can be substantially free of unreacted hemoglobin. As used herein, “unreacted hemoglobin” refers to hemoglobin that has not reacted or bound to another molecule and therefore has a low molecular weight. Unreacted hemoglobin, like cell-free hemoglobin, can extravasate out of circulation into the tissue space, thereby causing nitric oxide scavenging and subsequently vasoconstriction, systemic hypertension, and/or oxidative tissue injury. In some embodiments, the HBOC composition comprises less than 0.5% by weight unreacted hemoglobin, such as less than 0.1% by weight unreacted hemoglobin or less than 0.05% by weight unreacted hemoglobin.

In some embodiments, the composition can be substantially free of cell-free hemoglobin. As used herein, “cell-free hemoglobin” refers to hemoglobin molecules that have been separated from the red blood cells within which they originally occurred, often through the process of hemolysis. In some embodiments, the HBOC composition comprises less than 0.5% by weight cell-free hemoglobin, such as less than 0.1% by weight cell-free hemoglobin or less than 0.05% by weight cell-free hemoglobin.

In some embodiments, the composition can comprise less than 5% by weight hemoglobin species having a molecular weight of greater than 750 kDa, based on the total weight of the HBOC composition, such as less than 2.5% by weight hemoglobin species having a molecular weight of greater than 750 kDa, less than 1% by weight hemoglobin species having a molecular weight of greater than 750 kDa, less than 0.5% by weight hemoglobin species having a molecular weight of greater than 750 kDa, less than 0.1% by weight hemoglobin species having a molecular weight of greater than 750 kDa, or less than 0.05% by weight hemoglobin species having a molecular weight of greater than 750 kDa.

In some embodiments, the composition can comprise less than 5% by weight hemoglobin species having a hydrodynamic diameter of greater than 50 nm, based on the total weight of the HBOC composition, such as less than 2.5% by weight hemoglobin species having a hydrodynamic diameter of greater than 50 nm, less than 1% by weight hemoglobin species having a hydrodynamic diameter of greater than 50 nm, less than 0.5% by weight hemoglobin species having a hydrodynamic diameter of greater than 50 nm, less than 0.1% by weight hemoglobin species having a hydrodynamic diameter of greater than 50 nm, or less than 0.05% by weight hemoglobin species having a hydrodynamic diameter of greater than 50 nm.

In some embodiments, the composition can comprise less than 5% by weight hemoglobin species having a hydrodynamic diameter of greater than 0.1 microns, based on the total weight of the HBOC composition, such as less than 2.5% by weight hemoglobin species having a hydrodynamic diameter of greater than 0.1 microns, less than 1% by weight hemoglobin species having a hydrodynamic diameter of greater than 0.1 microns, less than 0.5% by weight hemoglobin species having a hydrodynamic diameter of greater than 0.1 microns, less than 0.1% by weight hemoglobin species having a hydrodynamic diameter of greater than 0.1 microns, or less than 0.05% by weight hemoglobin species having a hydrodynamic diameter of greater than 0.1 microns.

In some embodiments, the polymerized hemoglobin can be prepared by a process that includes polymerizing the hemoglobin, filtering the precursor solution by ultrafiltration against a first filtration membrane having a pore size that separates hemoglobin species having a molecular weight of less than 500 kDa from the polymerized hemoglobin; and filtering the precursor solution by ultrafiltration against a second filtration membrane having a pore size that separates hemoglobin species having a molecular weight of greater than 750 kDa from the polymerized hemoglobin

In some embodiments, the polymerized hemoglobin can be prepared by a process that includes polymerizing the hemoglobin, filtering the precursor solution by ultrafiltration against a first filtration membrane having a pore size that separates hemoglobin species having a molecular weight of less than 500 kDa from the polymerized hemoglobin; and filtering the precursor solution by ultrafiltration against a second filtration membrane having a pore size that separates hemoglobin species having a hydrodynamic diameter of greater than 50 nm from the polymerized hemoglobin.

In some embodiments, the polymerized hemoglobin can be prepared by a process that includes polymerizing the hemoglobin, filtering the precursor solution by ultrafiltration against a first filtration membrane having a pore size that separates hemoglobin species having a molecular weight of less than 500 kDa from the polymerized hemoglobin; and filtering the precursor solution by ultrafiltration against a second filtration membrane having a pore size that separates hemoglobin species having a hydrodynamic diameter of greater than 0.1 microns from the polymerized hemoglobin.

As used herein, “filtering” can include tangential-flow filtration. The term “tangential-flow filtration” refers to a process in which the fluid mixture containing the components to be separated by filtration is recirculated at high velocities tangential to the plane of the filtration membrane to reduce fouling of the filter. In such filtrations a pressure differential is applied along the length of the filtration membrane to cause the fluid and filterable solutes to flow through the membrane (e.g., filter). This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is continually processed downstream.

As used herein, “filtration membrane” refers to microporous barriers of, for example, polymeric, ceramic, or metallic materials which are used to separate dissolved materials (solutes), colloids, or find particular from solutions. A filtration membrane can be rated for retaining solutes have a specific molecular weight range from the molecular weight of one component in the solution to another component in the solution. By way of example, a filtration membrane can be rated for retaining polymerized hemoglobin with a molecular weight above that of a low molecular weight hemoglobin species (e.g., such as a membrane rated for retaining solutes have a molecular weight above 50 kDa).

In further embodiments, polymerizing hemoglobin includes adding to a Hb solution a crosslinker (e.g., glutaraldehyde) solution over a specified period of time. In some examples, polymerizing hemoglobin can comprise adding to a Hb solution a 0.75 wt. % glutaraldehyde solution in phosphate buffered saline (0.1 M, pH 7.4) at a 25: to 30:1 molar ratio, glutaraldehyde to Hb.

In some embodiments, the filtration membrane can be rated for retaining solutes having a molecular weight greater than the molecular weight of the low molecular weight hemoglobin species, thereby forming a retentate fraction including the polymerized hemoglobin and a permeate fraction including the low molecular weight hemoglobin species. As used herein, a “retentate fraction” refers to the fraction of solution that is unable to pass through the filtration membrane. In some embodiments, the retentate fraction can include polymerized hemoglobin. As used herein, a “permeate fraction” refers to the fraction of solution that permeates the filtration membrane. In some embodiments, the permeate fraction can include low molecular weight hemoglobin species. In other embodiments, the permeate fraction can include polymerized hemoglobin and low molecular weight hemoglobin species.

In some embodiments, ultrafiltration can include tangential-flow filtration. As used herein, the term “tangential-flow filtration” refers to a process in which the fluid mixture containing the components to be separated by filtration is recirculated at high velocities tangential to the plane of the filtration membrane to reduce fouling of the filter. In such filtrations a pressure differential is applied along the length of the filtration membrane to cause the fluid and filterable solutes to flow through the membrane (e.g., filter). This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is continually processed downstream.

In some embodiments, the composition can comprise from 1% by weight to 5% by weight albumin, based on total weight of the composition.

In some embodiments, the composition can have an osmolarity from 270 to 370 mOsm. In further embodiments, the composition can have an osmolarity from 270 to 290 mOsm, 290 to 310 mOsm, 310 to 330 mOsm, 330 to 350 mOsm, or 350 to 370 mOsm.

In some embodiments, the composition can have a viscosity from 1 cP to 10 cP at physiological temperature. In further embodiments, the composition can have a viscosity from 2 cP to 3 cP, 3 cP to 4 cP, or 4 cP to 4.5 cP.

In some embodiments, the composition can have a viscosity from 2 to 4 cP at physiological temperature. In further embodiments, the composition can have a viscosity from 2.1 to 3.1 cP, 3.1 to 3.3 cP, 3.3 to 3.5 cP, or 3.5 to 3.9 cP.

In some embodiments, the composition can have a colloid osmotic pressure from 1 mm Hg to 60 mm Hg. In further embodiments, the perfusion solution can have a colloid osmotic pressure from 14 mm Hg to 16 mm Hg, 16 mm Hg to 18 mm Hg, or 18 mm Hg to 20 mm Hg.

In some embodiments, the composition can have a colloid osmotic pressure from 10 mm Hg to 30 mm Hg.

In some embodiments, the polymerized hemoglobin can be synthesized using a molar ratio from 20:1 to 40:1 of glutaraldehyde to hemoglobin. In some embodiments, the polymerized hemoglobin can be synthesized using a molar ratio from 25:1 to 35:1 of glutaraldehyde to hemoglobin, or from 25:1 to 30:1 of glutaraldehyde to hemoglobin. In further embodiments, the molar ratio can be at least 1:1 (e.g., at least 2:1, at least 5:1, at least 10:1, at least 20:1, at least 30:1, at least 40:1, at least 50:1, at least 60:1, at least 70:1, at least 80:1, or at least 90:1). In certain embodiments, the molar ratio can be 100:1 or less (e.g., 90:1 or less, 80:1 or less, 70:1 or less, 60:1 or less, 50:1 or less, 40:1 or less, 30:1 or less, 20:1 or less, or 10:1 or less).

The range can vary between any of the minimum values described above to any of the maximum values described above. For example, the polymerized hemoglobin can be synthesized at a molar ratio that can range from 1:1 to 100:1 (e.g., from 1:1 to 10:1, 10:1 to 20:1, 20:1 to 30:1, 30:1 to 40:1, 40:1 to 50:1, 50:1 to 60:1, 60:1 to 70:1, 70:1 to 80:1, 80:1 to 90:1, 90:1 to 100:1).

In some embodiments, the partial pressure of oxygen at which 50% of the polymerized hemoglobin is saturated with oxygen can be from 1 mm Hg to 60 mm Hg. In further embodiments, the partial pressure of oxygen at which 50% of the polymerized hemoglobin is saturated with oxygen can be from 10 mm Hg to 12 mm Hg, 12 mm Hg to 14 mm Hg, 14 mm Hg to 16 mm Hg, 16 mm Hg to 18 mm Hg, or 18 mm Hg to 20 mm Hg. In some embodiments, the partial pressure of oxygen at which 50% of the polymerized hemoglobin is saturated with oxygen can be from 14 mm Hg to 16 mm Hg. As used herein, saturation with oxygen refers to the binding of heme in hemoglobin with oxygen molecules. The degree of oxygen saturation of hemoglobin is dependent on the number of heme units that are bound to oxygen (e.g., 50% saturation with oxygen means that half of the heme units are bound to oxygen molecules).

In some embodiments, the polymerized hemoglobin can exhibit an auto-oxidation rate at 37° C. of from 0.0020 to 0.0085 h⁻¹. In further embodiments, the polymerized hemoglobin can exhibit an oxidation rate of from 0.0020 to 0.0045 h⁻¹, 0.0045 to 0.0065 h⁻¹, or 0.0065 to 0.0085 h⁻¹. In some embodiments, the polymerized hemoglobin can exhibit an oxidation rate of from 0.0045 to 0.0065 h⁻¹.

In some embodiments, the perfusion solution can further include one or more of a metabolic suppressant agent. As used herein, “metabolic suppressant agent” refers to an agent used to suppress the metabolic demand that is required to keep organs and/or tissue at normothermic conditions during preservation for transplantation.

In some embodiments, the compositions described herein can be used to perfuse cardiac tissue.

In some embodiments, the composition can comprise a whole blood substitute. In certain embodiments, the composition can comprise polymerized hemoglobin, as described above, in combination with a clotting agent, a volume replacement solution, a drag reducing polymer, an anti-inflammatory agent, or a combination thereof.

The clotting agent can comprise, for example, tranexamic acid.

The volume replacement solution can comprise, for example, polymerized human serum albumin.

The drag reducing polymer can comprise, for example, polyethylene glycol.

The anti-inflammatory agent can comprise, for example, a steroid such as dexamethasone.

In some examples, the whole blood substitute can comprise from 25-200 mg/ml of the polymerized hemoglobin (PolyHb); from 25-200 mg/ml of a volume replacement solution, such as polymerized human serum albumin; from 1-50 mg/ml of a clotting agent, such as tranexamic acid; optionally from 0.05-2 mg/ml of a drag reducing polymer, such as polyethylene glycol; and optionally from 0.1-10 mg/ml of an anti-inflammatory agent, such as dexamethasone.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process. Only reasonable and routine experimentation will be required to optimize such process conditions.

### Example 1. Biophysical Properties and Preclinical Pharmacokinetics-Pharmacodynamics of Tangential Flow Filtration Facilitated Fractionated Polymerized Human Hemoglobin as a Red Blood Cell Substitute

### Overview

Polymerized human hemoglobin (PolyhHb) is being developed as a possible red blood cell (RBC) substitute for use in scenarios where blood is not available. However, product candidates that were tested in late phase clinical trials contained low molecular weight (LMW) hemoglobin (Hb) species (<500 kDa). Research suggests that LMW Hb species causes enhanced tissue distribution, vasoconstriction, and potential tissue injury, mediated by the reactivity of Hb iron within compartmentalized tissue spaces. Taken together, these physiological processes may have contributed to poor clinical trial outcomes.

In this example, we seek to improve the safety profile of tense quaternary state (T-state) PolyhHb via in vitro and in vivo screening of PolyhHb fractionated into 4 separate molecular weight (MW) brackets (50 kDa-300 kDa [PolyhHb-B1]; 100 kDa-500 kDa [PolyhHb-B2]; 500 kDa-750 kDa [PolyhHb-B3]; and 750 kDa-0.2 μm [PolyhHb-B4]) using a two-stage tangential flow filtration (TFF) purification process. In vitro screening showed that PolyhHb average molecular weight and diameter increased with increasing bracket size, while oxygen affinity, auto-oxidation kinetics and haptoglobin binding kinetics decreased with increasing bracket size. Initial screening of these four PolyhHb brackets in a 25% blood for PolyhHb exchange transfusion guinea pig model suggests that hypertension and tissue extravasation decreased with increasing bracket size. However, PolyhHb-B3 unexpectedly demonstrated extended circulatory pharmacokinetics, no renal tissue distribution, no aberrant blood pressure or cardiac conduction effects. Therefore, the PolyhHb-B3 bracket may offer the most appropriate material for further testing.

### Introduction

The most definitive clinical treatment for restoration of tissue oxygenation following blood loss is red blood cell (RBC) transfusion; however, RBCs may not be available or desirable to meet clinical transfusion needs. To address these issues, hemoglobin (Hb)-based oxygen (O₂) carriers (HBOCs) are actively being developed as RBC substitutes to replace blood transfusion demand. At first glance, a solution of acellular (cell-free) Hb may seem to be an appropriate HBOC, because of its ability to transport O₂. However, the protein is small enough (64 kDa) and can dimerize (32 kDa) at low concentrations to allow for extravasation through the endothelial lining of the blood vessel wall, depositing into the tissue space. In this environment, cell-free Hb is a reactive protein that readily depletes nitric oxide (NO), eliciting vasoconstriction and systemic hypertension. The iron contained in the heme prosthetic group of Hb also reacts with oxidants (e.g., hydrogen peroxide and superoxide) in the tissue space, which can contribute toward tissue injury. Therefore, to mitigate the cytotoxic effects of cell-free Hb, HBOCs need to be large enough to prevent tissue extravasation, while still maintaining Hb's native O₂ transport and allosteric properties. The most well studied HBOC consists of polymerized Hb (PolyHb)[13-18]. Hemolink® (Hemosol, Toronto, ON, Canada), Hemopure® (HbO₂ Therapeutics, Souderton, PA), and PolyHeme® (Northfield Laboratories Inc., Evanston, IL) are all commercial PolyHbs that were not approved by FDA following completion of pivotal Phase III clinical trials[19]. These PolyHbs are mostly composed of low molecular weight (LMW) Hb species (<500 kDa) and may exhibit adverse effects consistent with cell-free Hb.

To limit/reduce the adverse responses observed in some clinical trial patients treated with PolyHbs, one feasible approach is to remove LMW Hb species (<500 kDa) in solution. Work in our laboratory is focused on removing these LMW Hb species from the PolyHb intermediate product via tangential flow filtration (TFF), a scalable size-based separation technique. For example, in a 10% topload hamster window chamber model, vasoconstriction and systemic hypertension is inversely proportional to the size of the PolyHb molecule. Furthermore, in a hemodilution model, a high molecular weight (HMW) tense (T) quaternary state (T-state) PolyHb delivered O₂ to the tissue space and increased the tissue partial pressure of O₂ (pO₂) more than relaxed (R) quaternary state (R-state) PolyHb. In a 50% blood for PolyHb exchange transfusion (ET) model, we have also showed that T-state PolyHb decreased hypertension and oxidative tissue injury with increasing doses of PolyHb at the highest MW that can be prepared.

In another study, freshly synthesized HMW T-state PolyHb and 2 year old HMW T-state PolyHb were used to resuscitate animals from hemorrhagic shock (HS) and compared against fresh blood and stored blood. The results showed that resuscitation with PolyHb and blood restored blood pressure and cardiac function, but stored blood required a significantly larger transfusion volume to recover from HS compared with fresh blood and PolyHb. Stored blood transfusion also elevated markers of organ injury when compared with PolyHb and stored blood.

In another study, the T-state PolyHb was bracketed between 500 kDa and 0.2 μm in size, therefore removing the majority of LMW Hb species that elicited vasoconstriction, systemic hypertension, and renal tissue distribution, as well as any HMW Hb species over 0.2 μm in size that could signal the reticuloendothelial system (RES) to quickly clear the material from the circulation.

In this Example, we attempted to improve the fractionated T-state PolyHb bracket between 500 kDa and 0.2 μm by using TFF to further fractionate that bracket into sub-brackets: 500 kDa-750 kDa and 750 kDa-0.2 μm. The two new brackets were also compared against PolyHbs bracketed between 50 kDa-300 kDa and 100 kDa-500 kDa in terms of their biophysical properties, circulating pharmacokinetics (PK), tissue distribution, and hemodynamic and cardiac conduction response in a 25% blood for PolyHb ET guinea pig model. Through our analysis, we identified a PolyHb bracket (500 kDa-750 kDa) that unexpectedly optimizes PK parameters, tissue distribution, and/or hemodynamic response. The data from experimental results presented here provide proof-of-concept to pursue further studies with this PolyHb bracket. Future studies will rigorously interrogate the PolyHb in a HS guinea pig model with a pre-existing vascular dysfunction phenotype.

### Materials and Methods

Materials. Sodium dithionite (Na₂S₂O₄), glutaraldehyde (C₅H₈O₂) (70 wt %), sodium cyanoborohydride (NaCNBH₃), sodium lactate (NaC₃H₅O₃), N-acetyl-L-cysteine (NALC, C₅H₉NO₃S), and calcium chloride dihydrate (CaCl₂·2H₂O) were purchased from Sigma Aldrich (St. Louis, MO). Sodium chloride (NaCl), potassium chloride (KCl), sodium phosphate monobasic (NaH₂PO₄), sodium phosphate dibasic (Na₂HPO₄), and sodium hydroxide (NaOH) were purchased from Fisher Scientific (Pittsburgh, PA). Hollow fiber tangential flow filtration (TFF) modules S02-E050-05-N (modified polyethersulfone [mPES], 50 kDa pore size), S02-E100-05-N (mPES, 100 kDa pore size), S02-E300-05-N (mPES, 300 kDa pore size), S02-E500-05-N (mPES, 500 kDa pore size), S02-S500-05-N (polysulfone [PS], 500 kDa pore size), S02-E750-05-N (mPES, 750 kDa pore size), and S02-P20U-05-N(polyethersulfone [PES], 0.2 μm pore size) were purchased from Repligen (Rancho Dominguez, CA). The Liqui-Cel™ MM Series G 543 Membrane Contactor was purchased from 3M (St. Paul, MN). Expired human red blood cell (RBC) units were acquired from Transfusion Services at the Wexner Medical Center (Columbus, OH), Canadian Blood Services (Ottawa, Canada), and Zen-Bio Inc. (Durham, NC).

hHb Polymerization. Human hemoglobin (hHb) was purified via TFF as previously described from expired human RBC units. The purified hHb was cross-linked with glutaraldehyde and the resulting polymerized hHb (PolyhHb) purified via TFF as previously described. In summary, 30 g of purified hHb was added to a 2 L reaction vessel and diluted to 20 mg/mL with phosphate buffered saline (PBS) at pH 7.40. The system was maintained at 37° C. using a hot plate. The hHb solution was pumped through a gas-liquid contactor recirculation loop to deoxygenate the solution with nitrogen gas as the sweep gas to lock the protein in the T-state. The reactor headspace was also filled with nitrogen gas to maintain anaerobic conditions in the reactor system. The partial pressure of O₂ (pO₂) of the hHb solution was measured after 30 minutes of gas-liquid assisted deoxygenation using a RAPIDLab 248 blood gas analyzer (Siemens, Munich, Germany). Once the pO₂ reached <20 mm Hg, a bolus injection of 300 mg sodium dithionite (Na₂S₂O₄) dissolved in 50 mL of degassed PBS was introduced into the system. After 10 minutes, the pO₂ was measured and if a reading of 0.0 mm Hg was obtained, the polymerization process was initiated via addition of glutaraldehyde.

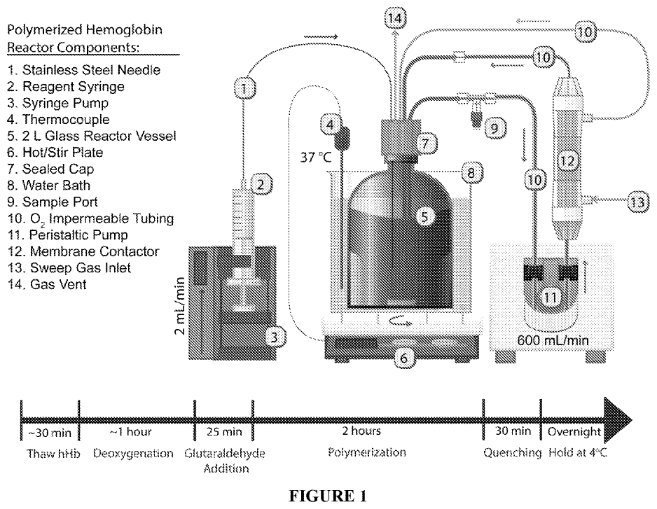

The molar ratio of glutaraldehyde (chemical cross-linker) to hHb was varied depending on the desired PolyhHb molecular weight (MW) bracket. For PolyhHb MW brackets B1 (50-300 kDa), B2 (100-500 kDa), B3 (500-750 kDa), and B4 (750 kDa-0.2 μm), the molar ratio of glutaraldehyde to hHb was 10:1, 25:1, 26.5:1, and 30:1, respectively. Glutaraldehyde was prepared as a solution in 50 mL of degassed PBS and fed to the reactor at a rate of 2 mL/min. After two hours of reaction time, the system was quenched with a 7:1 molar ratio of sodium cyanoborohydride to glutaraldehyde dissolved in 50 mL of PBS. The solution was reacted for an additional 30 minutes and was then stored at 4° C. overnight. The reactor schematic and synthesis timeline are shown in FIG. 1.

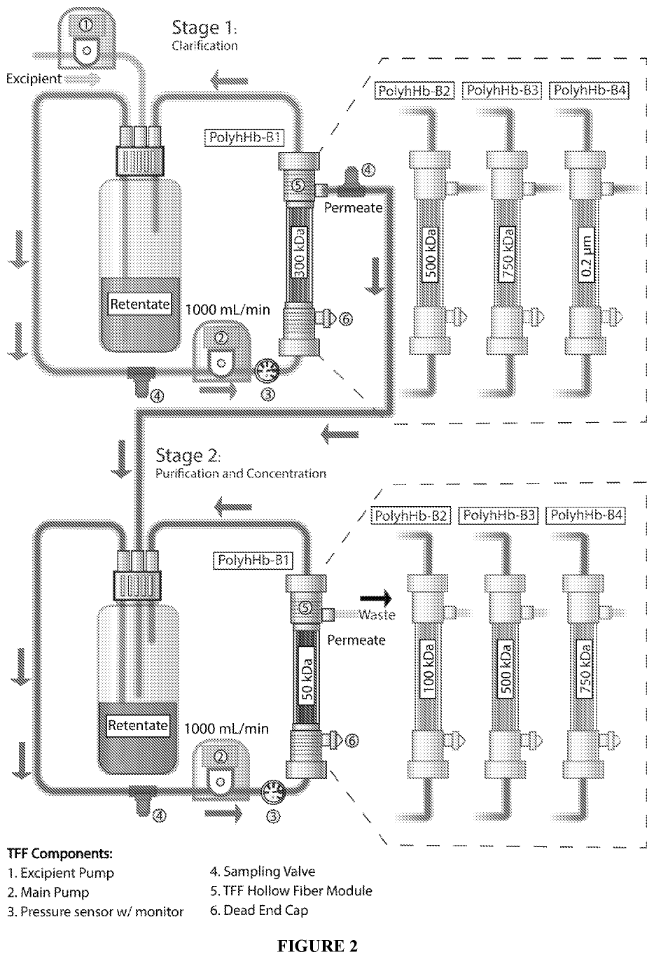

PolyhHb Fractionation and Purification. As shown in FIG. 2, each PolyhHb bracket was purified using two TFF modules based on the desired MW range of the bracket. The procedure for fractionation and purification was modified from the protocol described in the literature[29,30]. The first filtration stage was used to removed large MW polymerized material. For brackets PolyhHb-B1, PolyhHb-B2, PolyhHb-B3, and PolyhHb-B4, the upper MWCO of the TFF module was set to 300 kDa mPES, 500 kDa mPES, 750 kDa mPES, and 0.2 μm PES, respectively. In the second stage, the PolyhHb solution was excipient exchanged over a low MWCO TFF module with a modified Ringer's lactate solution to remove free hHb, unreacted reagents, and low MW species from the final product. For brackets PolyhHb-B1, PolyhHb-B2, PolyhHb-B3, and PolyhHb-B4, the lower MWCO was set to 50 kDa mPES, 100 kDa mPES, 500 kDa PS, and 750 kDa mPES, respectively. The product was excipient exchanged for eight diacycles, or until the permeate concentration of hHb was less than 0.5 mg/mL. Following the completion of the diafiltration process, the final product was concentrated over the low MWCO TFF module to 10 g/dL. The final concentration of total protein and methemoglobin (metHb) was measured via UV-visible spectrometry (Olis Inc., Bogart, GA) using the cyanomethemoglobin method[31]. The final product was stored at −80° C. until use.

SEC-HPLC Analysis. The MW distribution of hHb and PolyhHb brackets were measured using size exclusion chromatography (SEC) on a high-performance liquid chromatography (HPLC) system. The separation was performed using an Ultimate 3000 system with an SEC-1000 column (ThermoFisher Scientific, Waltham, MA). The absorbance was measured at 413 nm to monitor the Soret peak of hHb/PolyhHb. For hHb and PolyhHb brackets B1 and B2, the number of replicates was n=3. For PolyhHb brackets B3 and B4, the number of replicates was n=7 and n=4, respectively.

Hydrodynamic Diameter via DLS. The hydrodynamic diameter of hHb and PolyhHb brackets were measured via dynamic light scattering (DLS) on a BI-200SM Research Goniometer (Brookhaven Instruments Corp., Holtsville, NY) following the procedure described in literature[32]. The number of replicates for hHb, and PolyhHb brackets B1, B2, B3, and B4 were n=3, n=5, n=5, n=13, and n=5, respectively.

Sodium Dodecyl Sulfate Polyacrylamide Gel Electrophoresis (SDS-PAGE). SDS-PAGE gel electrophoresis was used to assess the presence of low MW and free hHb species in the PolyhHb brackets. Samples of unmodified hHb and each bracketed PolyhHb were prepared at 1 mg hHb/mL and mixed in a 1:1 ratio with Tris Glycine SDS sample buffer (Novex, Carlsbad, CA). A Novex WedgeWell 4-20% Tris-Glycine Gel (Invitrogen, Waltham, MA) was used and loaded with 20 μL of each sample solution. The gel was run for 40 minutes at 225 V and stained with Coomassie Blue staining solution for 30-45 minutes. The gel was then destained overnight with a solution of 3:1:6 v/v/v methanol:acetic acid:DI water mixture.

O₂ Equilibrium Curve. The Hemox Analyzer (TCS Scientific Corp., New Hope, PA) was used to measure the O₂ equilibrium curve (OEC) for hHb and PolyhHb brackets following the procedure described by Palmer et al.[30]. The OEC data was fit to the Hill equation to regress the cooperativity coefficient (n_{H}) and pO₂ at which 50% of the hHb or PolyhHb is saturated with O₂ (i.e. P₅₀)[33]. The number of replicates that were characterized for hHb, and PolyhHb brackets B1, B2, B3 and B4 were n=3, n=5, n=5, n=12, and n=5, respectively.

Stopped-Flow Kinetics. A SX-20 micro-volume stopped-flow apparatus (Applied Photophysics, Leatherhead, UK) was used to measure the O₂ offloading kinetics and the haptoglobin (Hp) binding kinetics of hHb and PolyhHb brackets. The measurement of O₂ offloading kinetics followed the protocol described by Rameez et al.[34] For O₂ offloading kinetics, the absorbance at 437.5 nm was monitored when 12.5 μM (heme-basis) of hHb/PolyhHb in PBS was mixed with a 1.5 M solution of sodium dithionite in degassed PBS. The average of the kinetic traces was fit to an exponential function using the Applied Photophysics software to regress the first order O₂ dissociation rate constant (k_{O2,off}). The number of replicates that were characterized for hHb, PolyhHb brackets B1, B2, B3 and B4 were n=3, n=5, n=5, n=13, and n=5, respectively.

The measurement of Hp binding kinetics followed the protocol described by Gu et al.[35]. In summary, the Hp binding kinetics measures the change in fluorescence that occurs when Hp binds hHb/PolyhHb as measured at an emission wavelength of 310 nm via excitation at 285 nm. The following concentrations of hHb or PolyhHb in PBS (hHb tetramer basis), 5 μM, 10 μM, 15 μM, and 20 μM, were rapidly mixed with Hp (0.25 μM, hHb-binding basis). The average of the kinetic traces was fit to an exponential function, and a pseudo-first order rate constant was regressed for each concentration. The apparent rate constants were plotted against hHb/PolyhHb concentration and fit to a linear function to regress the second order Hp binding rate constant (k_{Hp-Hb}) in units of μM⁻¹·s⁻¹. The number of replicates that were characterized for hHb, and PolyhHb brackets B1, B2, B3 and B4 were n=3, n=5, n=3, n=8, and n=5, respectively.

Auto-Oxidation Kinetics. UV-visible spectroscopy was used to measure the auto-oxidation kinetics of hHb and PolyhHb brackets. The hHb or PolyhHb bracket was diluted to 0.775 mM (heme basis) using PBS (pH 7.40). The solution was maintained for 24 hours at 37° C. The absorbance at 630 nm was monitored over a 24-hour period. The percent of hHb in the oxygenated ferrous state as a function of time was analyzed assuming first order rate kinetics to solve for the first order auto-oxidation rate constant (kₒₓ) in units of s⁻¹. The number of replicates that were characterized for hHb, and PolyhHb brackets B1, B2, B3 and B4 were n=3, n=3, n=3, n=7, and n=4, respectively.

Animal Experiments. All studies were approved by the Institutional Animal Care and Use Committee (IACUC) at the University of Maryland Baltimore (protocol #0320006). Surgery and catheter placement in guinea pigs was performed as previously described for exchange transfusion studies with chemically modified hemoglobin preparations[26,36].

Exchange Transfusion. The body weight of guinea pigs evaluated in this study was 663.3±58.7 grams. A 25% exchange transfusion volume (ETV) was estimated using the formula ETV=70 mL/kg×guinea pig body weight (kg)×0.25 (25% ETV)[37]. For this study the ETV ranged between 11-13 mL. All bracketed PolyhHb fractions were normalized to a concentration of 100 mg/mL using 0.9% NaCl prior to transfusion. Exchange transfusion (ET) was performed by simultaneously infusing PolyhHb-(B1, B2, B3 or B4) at a rate of 1 mL/min, while simultaneously withdrawing 1 mL/min of blood until reaching complete infusion of the PolyhHb volume.

Blood/Tissue Collection. A 100 μL sample of blood was collected before ET, immediately after ET (0 hours), and at 1, 2, 4, 8, 10, 24, 30, and 48 hours following ET. Blood was collected until plasma concentrations reached approximately 90% of the maximum plasma concentration, Cₘₐₓ (30 hours for B1 and B2, 48 hours for B3 and B4). This allowed us to harvest tissue to assess PolyhHb tissue distribution when plasma concentrations in each group were similar, given that PolyhHb-B3 and -B4 persisted longer than PolyhHb-B1 and -B2 in circulation. Immediately after the final blood collection, animals were humanely euthanized and exsanguinated of blood. Organs (kidneys (left, right), liver (4 lobes), spleen, heart, and lungs) were washed with cold 0.9% NaCl. The tissues from each animal were separated with half stored in 10% formalin (24 hour fixation and 70% isopropanol storage) and half snap frozen in liquid nitrogen and stored at −80° C.

Pharmacokinetic Analysis. Pharmacokinetic (PK) parameters were estimated for PolyhHb concentrations over time to evaluate the exposure characteristics of each bracketed formulation as a function of molecular size (PolyhHb-B1, n=8; PolyhHb-B2, n=6; PolyhHb-B3, n=6; PolyhHb-B4, n=6). A non-compartmental analysis was performed using Phoenix 64 WinNonlin, version 8.2.0 (Certara, Princeton, NJ) to determine PK parameter estimates. The maximum plasma concentration (Cₘₐₓ) was taken as the concentration in plasma obtained at the end of ET. The area under the plasma concentration time curve (AUC)₀₋₂₈, was estimated using the linear trapezoidal rule to the last measurable concentration (AUC_{0-Clast}). Extrapolation to infinity (AUC_{Clast-∞}) was accomplished by dividing the last measurable plasma concentration (Cₗₐₛₜ) by the negative value of the terminal slope (k) of the log-linear plasma concentration-time curve. Thus, AUC_{0-∞} is equal to the sum of AUC and AUC_{Clast-∞} and plasma clearance (CL) as dose divided by AUC_{0-∞}. Additional parameters were calculated as follows: AUMC volume of distribution (V_{c}) as dose divided by Cₘₐₓ, and half-life (t_{1/2}) as the product of ln(2) and mean residence time (MRT).

Perls Iron Staining with DAB Intensification. Tissue sections were dewaxed-hydrated, incubated (45 min, RT) in 0.3 M HCl (ACROS Organics, Geel, Belgium) and 2.5% (w/v) potassium ferrocyanide trihydrate (ThermoFisher, Waltham, MA, USA), and washed with distilled water. Sections were then incubated (30 min, RT) in methanol containing 0.3% H₂O₂(LabChem, Zelienople, PA, USA) and 0.01 M NaN₃ (Sigma-Aldrich, St. Louis, MO, USA). Sections were washed in 0.1 M phosphate buffer (pH 7.4) and incubated (3 min, RT) with 3,3′-diaminobenzidine (DAB) and H₂O₂(SIGMAFAST™, Sigma-Aldrich, St. Louis, MO, USA). Sections were then counterstained in hematoxylin (Gil no. 2, Fisher Scientific, Waltham, MA, USA).

Immunohistochemistry. Heat-mediated antigen retrieval was performed using pH 6.0 citrate buffer (Sigma-Aldrich, St. Louis, MO, USA). The solution was brought to a boil, and then allowed to cool for 30 min. Once cool, sections were incubated (60 min, RT) with 3% horse serum (ThermoFisher, Waltham, MA, USA). Sections were then incubated (overnight, 4° C.) with an antibody against heme oxygenase-1 (HO-1; 1:200) (Primary Ab: HO-1/HMOX1 Polyclonal antibody (Cat #10701-1-AP, Proteintech, Rosemont, IL, USA). To block any endogenous peroxidase activity, sections were then incubated (10 min, RT) with 3% H₂O₂(LabChem, Zelienople, PA, USA). Sections were then incubated (30 min, RT) with a biotinylated secondary antibody (1:300) (Secondary Ab: Goat anti-Rabbit IgG (H+L), Biotin (Cat #31820, Invitrogen Invitrogen, Waltham, MA, USA). Sections were then incubated (30 min, RT) with avidin and biotinylated horseradish peroxidase (VECTASTAIN® Elite® ABC Kit, Vector Laboratories, Newark, CA, USA), and incubated (3 min, RT) with DAB and H₂O₂(SIGMAFAST™, Sigma-Aldrich, St. Louis, MO, USA). Lastly, sections were then counterstained in hematoxylin (Gil no. 2, ThermoFisher, Waltham, MA, USA).

Brightfield microscope images were obtained using a DM4 B microscopy system (Leica Biosystems, Wetzlar, Germany) for image analysis at 4×, 20×, and 63× magnifications. The visualization and assessment of renal and hepatic iron distribution was performed by image scanning of tissue sections after Perls iron staining. All tissue images were analyzed using ImageJ software[38].

Hemodynamic Measurements. Guinea pig vital signs were recorded using a PowerLab 16/35 system (ADInstruments, Sydney, Australia), using an electrocardiogram (ECG) lead ii, a blood pressure transducer, and a rectal temperature probe. Data was processed using LabChart v8.1.21 (ADInstruments, Sydney, Australia). Rectal temperature and mean arterial pressure (MAP) were calculated by averaging vital signs over a 90 second interval. Heart rate, systolic blood pressure, and diastolic blood pressure values were calculated from the ECG and arterial pressure channels by the software over the same interval. Pulse pressure was calculated directly as the difference between systolic and diastolic pressures. Data was processed in Prism (v9.4.1). Areas under curves were compared using an ordinary one-way ANOVA with multiple comparisons using Tukey's correction and a family-wise α=0.05.

Statistical Analysis. All data was presented as the mean±standard deviation. Statistical analyses of the data were performed using JMP (version JMP Pro 15, JMP Statistical Discovery LLC, Cary, NC) using student's paired t-tests. For all tests, p<0.05 was considered statistically significant. * denotes a statistically significant difference (p<0.05) compared to the hHb control. † denotes a statistically significant difference (p<0.05) compared to low MW PolyhHb (PolyhHb-B1 and/or PolyhHb-B2). Denotes a statistically significant difference (p<0.05) compared to high MW PolyhHb (PolyhHb-B3 and/or PolyhHb-B4). For guinea pig studies, hemodynamic and pharmacokinetic parameter data were analyzed in GraphPad Prism 9.1.1 (San Diego, California, USA) using a One-way ANOVA and Tukey's corrected multiple comparisons test at α=0.05. All data is represented as the mean±SD of individual values. For all tests, p<0.05 is considered statistically significant and represented as: *p<0.05, **p<0.01, ***p<0.001, ****p<0.0001.

Table 1. Biophysical properties of hHb and PolyhHb brackets, including properties related to O₂ binding, size, and Hp binding kinetics. Measured parameters are reported as the average±standard deviation. * denotes a statistically significant difference (p<0.05) compared to the hHb control. † denotes a statistically significant difference (p<0.05) compared to low MW PolyhHb (PolyhHb-B1 and/or PolyhHb-B2). ‡ denotes a statistically significant difference (p<0.05) compared to high MW PolyhHb (PolyhHb-B3 and/or PolyhHb-B4).

TABLE 1

| | hHb | PolyhHb-B1 | PolyhHb-B2 | PolyhHb-B3 | PolyhHb-B4 |
|---|---|---|---|---|---|
| Glutaraldehyde:hHb | 0:1 | 10:1 | 25:1 | 26.5:1 | 30:1 |
| Molar Ratio | | | | | |
| Stage 1 Filter MWCO | N/A | 300 kDa mPES | 500 kDa mPES | 750 kDa mPES | 0.2 μm PES |
| Stage 2 Filter MWCO | N/A | 50 kDa mPES | 100 kDa mPES | 500 kDa PS | 750 kDa mPES |
| Average MW (kDa) | 64.7 ± 0.2 | 105.8 ± 5.8 | 351.5 ± 91.1*† | 586.2 ± 100.3*† | 1204.9 ± 122.7*†‡ |
| Effective Diameter (nm) | 5.0 ± 0.5 | 7.4 ± 1.2 | 11.7 ± 2.8*† | 19.9 ± 2.3*† | 32.1 ± 4.9*†‡ |
| P₅₀ (mm Hg) | 13.29 ± 1.00 | 17.50 ± 5.45 | 34.13 ± 8.81*† | 32.77 ± 6.91*† | 40.22 ± 6.71*†‡ |
| Hill Coefficient, n_{H} | 2.45 ± 0.35 | 1.10 ± 0.13* | 0.99 ± 0.02* | 0.97 ± 0.10* | 0.92 ± 0.04*† |
| k_{O2 ,off} (s⁻¹) | 30.15 ± 2.51 | 31.86 ± 2.51 | 46.18 ± 8.22*† | 46.79 ± 4.13*† | 47.74 ± 10.17*† |
| k_{Hp-Hb} (μM⁻¹ s⁻¹) | 0.0995 ± 0.0070 | 0.0672 ± 0.0101* | 0.0323 ± 0.0138*† | 0.0047 ± 0.0016*† | 0.0071 ± 0.0027*† |
| k_{ox, fast} (hr⁻¹) | 0.0221 ± 0.008 | 0.0134 ± 0.0028 | 0.0108 ± 0.0036 | 0.0171 ± 0.0068† | 0.0195 ± 0.0056 |
| k_{ox, slow} (hr⁻¹) | N/A | 0.0107 ± 0.0042* | 0.0086 ± 0.0057* | 0.0127 ± 0.0056* | 0.0132 ± 0.0067* |

### Results and Discussion

In this Example, hHb was polymerized at various glutaraldehyde to hHb molar ratios to generate PolyhHb species in each of the 4 MW brackets: PolyhHb-B1 (50-300 kDa), PolyhHb-B2 (100-500 kDa), PolyhHb-B3 (500-750 kDa), and PolyhHb-B4 (750 kDa-0.2 μm). The final synthesized product was clarified to remove species >0.2 μm and large MW PolyhHb species, diafiltered, and concentrated over a series of TFF modules of various sizes (Table 1). The final four brackets, PolyhHb-B1, PolyhHb-B2, PolyhHb-B3, and PolyhHb-B4, were characterized to determine the effects that polymer size (hydrodynamic diameter and MW distribution) have on PolyhHb biophysical parameters including O₂ equilibria and offloading, auto-oxidation kinetics, and Hp binding kinetics. All parameters were compared against unmodified hHb and between the high MW (PolyhHb-B3 and PolyhHb-B4) and low MW (PolyhHb-B1 and PolyhHb-B2) brackets. The values of those parameters are listed in Table 1.

TFF module MWCOs of the respective PolyhHb brackets were selected based on the available filter modules (pore size and membrane material) from Repligen. The PolyhHb-B1 bracket consisted of a low-end MWCO of 50 kDa to remove some unmodified hHb (64.8±1.8 kDa) from the product. The TFF module itself is composed of modified polyethersulfone (mPES), a hydrophilic material, that facilitates the passage of molecules slightly above the MWCO cut-off listed by Repligen (i.e., unmodified hHb) through the membrane. The PolyhHb-B2 bracket had a low MWCO TFF module of 100 kDa mPES, that was available from Regligen. This filter, similar that of the 50 kDa module in PolyhHb-B1, allowed materials greater than 100 kDa to permeate through the membrane and kept the average MW of the final product between 300-500 kDa. The PolyhHb-B2 bracket represents the low MW species that are typically present in commercialized HBOCs polymerized with glutaraldehyde, such as Hemopure® (HBOC-201, 130-500 kDa[40]) (Biopure Corp., Cambridge, MA) and PolyHeme® (130-250 kDa[40]) (Northfield Laboratories Inc., Evanston, IL)[41,42]. The PolyhHb in this MW bracket have been shown to elicit hypertension and other oxidative events because of extravasation or interaction with the endothelium of the blood vessels[22,43,44]. Finally, the PolyhHb-B3 and PolyhHb-B4 brackets represent high MW PolyhHb species splitting the established MW ranges for PolyhHb produced from our group.

The molar ratio of glutaraldehyde to hHb was experimentally screened to determine which molar ratio generated the highest yield of PolyhHb within the designated MW bracket. Initial cross-linking ratios were selected based on the average MW of PolyhHb synthesized at different cross-link densities. Based on the final yield of PolyhHb and whether the majority of PolyhHb lost was in stage 1 or stage 2 of the PolyhHb purification process, the cross-linking ratio was appropriately adjusted. Synthesis of PolyhHb-B1, PolyhHb-B2, and PolyhHb-B4 had yields greater than 40%, thus requiring less replicates to generate the total material necessary for in vivo evaluation and contributing to the lower number of replicates. PolyhHb-B3 consistently had very low yields (<25%) and required more replicates to achieve the amount of material needed for in vivo studies.

### SEC-HPLC

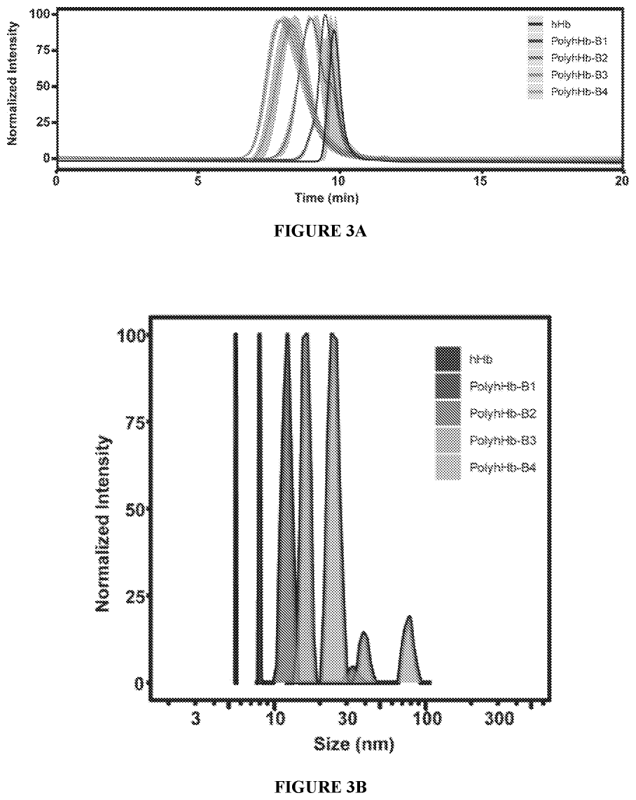

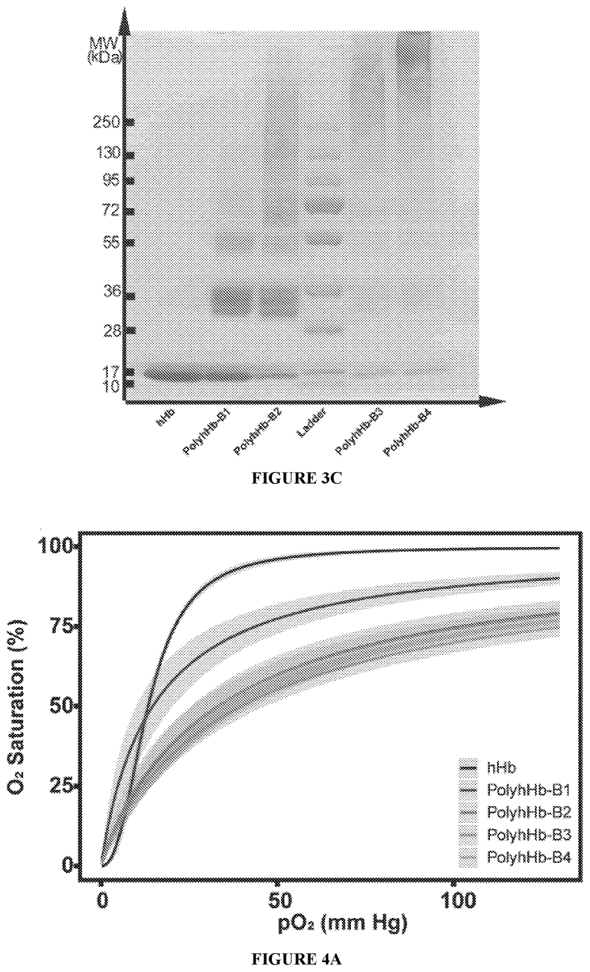

SEC-HPLC was used to estimate the MW distribution of the PolyhHb brackets. FIG. 3A shows the elution time for the four PolyhHb brackets compared to hHb. In Table 1, the elution time distribution of each PolyhHb bracket was used to calculate the average MW via a deconvolution R Studio script[28]. PolyhHb-B1, -B2, -B3, and -B4 were found to have an average elution time of 9.49, 8.98, 8.49, and 7.96 min, demonstrating increasing MW with increasing cross-link density. All PolyhHbs exhibited faster elution times compared to unmodified hHb (9.73 min). The average MW of each PolyhHb bracket is statistically different from the unmodified hHb control and from each of the other brackets (p<0.05), with the exception of PolyhHb-B1 and hHb (p>0.05). PolyhHb-B4 exhibited the largest average MW, which decreased with smaller PolyhHb brackets.

Unmodified cell-free hHb in circulation is able to extravasate through the pores of the blood vessel walls and subsequently elicits vasoconstriction and oxidative tissue injury[7]. To minimize the quantity of cell-free hHb in the final PolyhHb product, excipient exchanges over the lower MWCO TFF module was performed. The SEC-HPLC elution curve of each PolyhHb bracket can provide insight into the relative quantities of unmodified hHb present in the final product. For PolyhHb-B4 and PolyhHb-B3, there is a tail on the right-hand side of the SEC-HPLC elution curve, which is indicative of the presence of lower MW PolyhHb species and small quantities of unmodified hHb in the product. PolyhHb-B2 has a similar trend but with a shoulder, rather than a tail. Finally, PolyhHb-B1 has the most overlap with the peak corresponding to unmodified hHb, therefore demonstrating the presence of the largest amount of free hHb in solution. Hence, as the cross-link density increases, the amount of unmodified hHb in the PolyhHb bracket decreases with PolyhHb-B4 having the least amount. This observation from SEC-HPLC analysis was further confirmed with SDS-PAGE analysis.

Commercial HBOCs, PolyHeme® and Hemopure®, have similar size distributions to PolyhHb-B2. These HBOCs have been reported as having 1% and 5% of final product species less than 64 kDa, respectively[45]. Although this quantity is small, most species are still between 100-500 kDa and therefore are still likely to extravasate through the pores lining the blood vessel wall into the tissue space[7].

### DLS

The effective hydrodynamic diameter of the PolyhHb brackets and unmodified hHb was measured via DLS. Unmodified hHb has an effective diameter of 5.0±0.5 nm. The hydrodynamic diameter of the PolyhHb bracket increased with increasing cross-link density, with PolyhHb-B4 yielding the largest PolyhHb species at 32.1±4.9 nm and polymerized at a glutaraldehyde to hHb molar ratio of 30:1. The distribution of sizes is shown in FIG. 3B and their respective average diameters are listed in Table 1. When compared individually, the materials in the large MW brackets, PolyhHb-B3 and PolyhHb-B4, are significantly larger than unmodified hHb and the low MW bracketed species, PolyhHb-B1 and PolyhHb-B2 (p<0.05). PolyHeme® and Hemopure® have measured diameters of 7.9 nm and 8.4 nm[46] which is similar to PolyhHb-B1 (7.4±1.2 nm). The small average hydrodynamic diameters of these species are consistent with the presence of a large fraction of low MW species (<500 kDa), including unmodified hHb (64 kDa), which would lead to extravasation and subsequent deleterious effects in vivo[47].

### SDS-PAGE

To further confirm the lower range of the PolyhHb size distribution measured via SEC-HPLC, the materials were analyzed via SDS-PAGE. FIG. 3C shows the gel for unmodified hHb and each PolyhHb bracket. The band on the gel at ~16 kDa corresponds to individual α/β globins. The bands at ~30 kDa correspond to cross-linked ap dimers. Proceeding from hHb to PolyhHb-B4, the intensity of the unmodified hHb bands reduces, indicating the presence of successively less unmodified hHb with larger MW PolyhHb brackets. Another key feature is the presence of smeared bands in the high MW region of the gel. The smeared bands are characteristic of the polydisperse distribution of PolyhHb[35]. The SDS-PAGE used for this analysis was not designed for the migration and separation of species >250 kDa, hence the presence of the smearing effect. From PolyhHb-B1 to PolyhHb-B4, the intensity of the smear moves closer to the top of the gel, indicating an increase in average MW from PolyhHb-B1 to PolyhHb-B4, which are consistent with the results from SEC-HPLC and DLS analysis.

### OEC

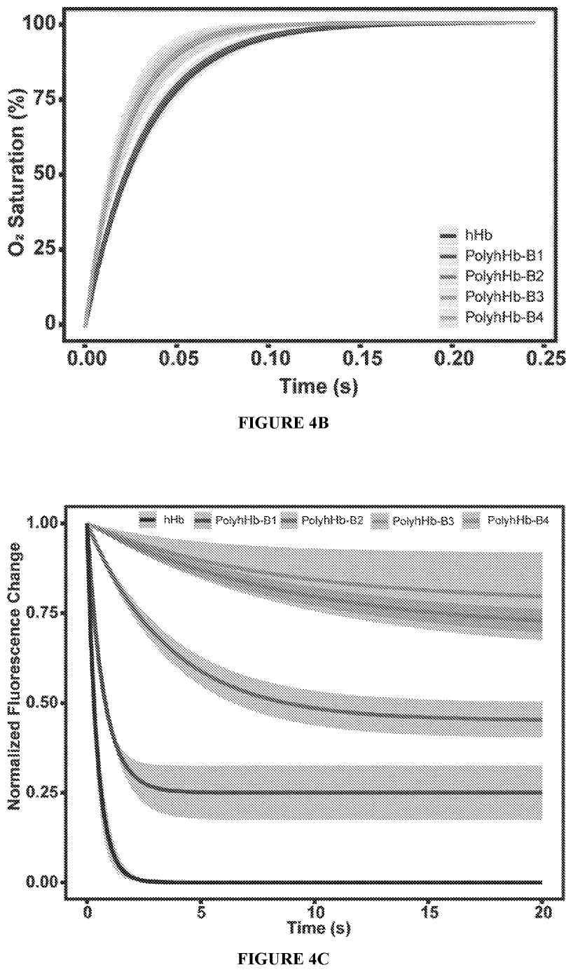

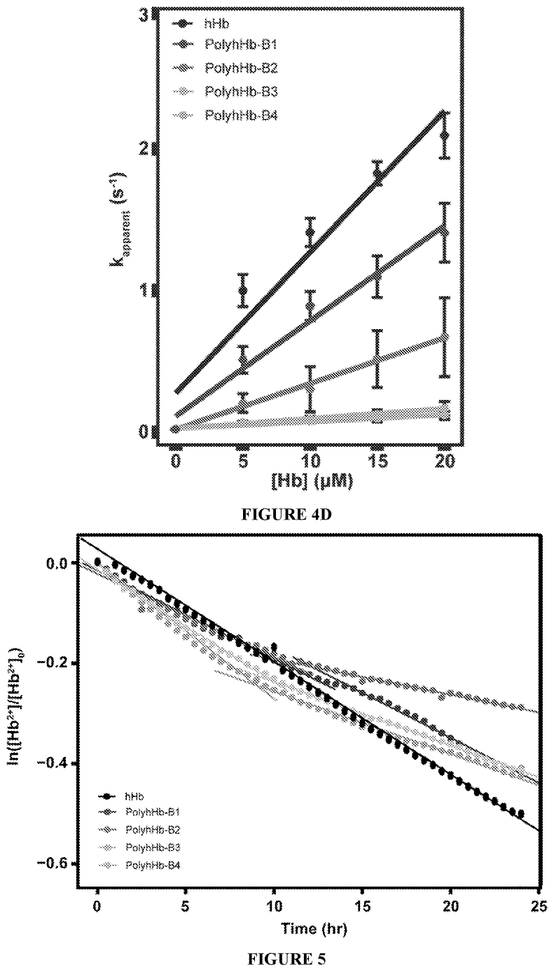

To evaluate the O₂ equilibrium of unmodified hHb and PolyhHb brackets, the O₂ equilibrium curve (OEC) was measured using a HEMOX analyzer. The OECs were fit to the Hill equation to regress the P₅₀ and n_{H}[33]. FIG. 4A shows the OECs for unmodified hHb and bracketed PolyhHb. The values for the P₅₀ and n_{H} are listed in Table 1. The P₅₀ of PolyhHb-B2, PolyhHb-B3, and PolyhHb-B4 are significantly greater than the P₅₀ of PolyhHb-B1 and unmodified hHb (p<0.05).

Prior to the cross-linking reaction, the starting solution of hHb was completely deoxygenated to a pO₂ of 0 mm Hg. This was done to lock the PolyhHb in the T-state, which exhibits a lower O₂ affinity and easier O₂ offloading compared to hHb[8,48]. The high P₅₀ of PolyhHb-B2, PolyhHb-B3, and PolyhHb-B4 are greater than 25 mm Hg and are consistent with previous high MW T-state PolyhHbs synthesized in our lab[22,28,39]. The P₅₀ of 17.50 mm Hg for PolyhHb-B1 is indicative of the presence of a significant fraction of tetrameric hHb species (α₂β₂), which lowered the P₅₀ of PolyhHb-B1 to a value between that of T-state (>25 mm Hg) and unmodified hHb (13.29±1.00 mm Hg). Commercial HBOCs, PolyHeme® and Hemopure® are similar in size to PolyhHb-B2 and are also locked in the T quaternary state during polymerization. The P₅₀ of PolyHeme® and Hemopure® were 31.34±0.80 mm Hg and 34.28±0.77 mm Hg[40], respectively, which is consistent with the P₅₀ of PolyhHb-B2 and -B3, which were not significantly different from one another (p>0.05).

Once hHb is cross-linked and polymerized in the T quaternary state, cooperative motions of the globin chains are restricted[49]. This is reflected in a n_{H} of 1.10±0.13, 0.99±0.02, 0.97±0.10, and 0.92±0.04 for PolyhHb-B1 to PolyhHb-B4, respectively. The Hill coefficient for all brackets is close to a value of one, indicative of noncooperative O₂ binding. The cooperative O₂ binding nature of unmodified hHb is represented by a Hill coefficient of 2.45±0.35, which gives rise to the characteristic sigmoidal shape of the OEC curve[33]. The Hill coefficient for all PolyhHb brackets is significantly different than that of unmodified hHb (p<0.05). The commercial polymerized HBOCs have reduced cooperativity with Hill coefficients for PolyHeme® and Hemopure® of 1.7 and 1.4, respectively[45].

### O₂ Offloading

T-state PolyhHb has a lower O₂ affinity and more rapidly offloads O₂ to surrounding tissues compared to hHb[19,50]. FIG. 4B shows the O₂ offloading kinetics of unmodified hHb and all PolyhHb brackets. From Table 1, PolyhHb-B2, PolyhHb-B3, and PolyhHb-B4 were significantly faster at offloading O₂ than PolyhHb-B1 and unmodified hHb (p<0.05). The trends observed with the higher MW PolyhHb brackets is consistent with the fact that T-state PolyhHb releases O₂ more readily than unmodified hHb[23,28]. The similarity in rate constants of PolyhHb-B1 (31.86±2.51 s⁻¹) and unmodified hHb (30.15±2.51 s⁻¹) is most likely a result of the presence of unmodified hHb in the PolyhHb-B1 product, thus reducing the average overall rate constant of O₂ offloading. PolyHeme® and Hemopure® have reported O₂ offloading constants of 84.0±0.3 s⁻¹ and 52.2±0.2 s⁻¹, respectively[40]. These values reflect an extremely low affinity for O₂ and therefore rapid O₂ offloading.

### Hp Binding

The deleterious effects of cell-free hHb in the circulation can be mitigated via binding to the plasma protein haptoglobin (Hp)[51]. FIG. 4C shows the pseudo-first order binding kinetics for Hp binding to hHb and each PolyhHb bracket. FIG. 4D plots the pseudo-first order binding rate constant as a function of concentration of PolyhHb or hHb. The data was fit to a linear function to regress the second order rate constant, k_{Hp-Hb}, for each PolyhHb bracket and hHb, listed in Table 1. PolyhHb-B3 and PolyhHb-B4 have relatively low rate constants (slope) of 0.0047±0.0017 μM⁻¹ s⁻¹ and 0.0071±0.0027 M⁻¹ s⁻¹, respectively, indicating that they do not readily bind to Hp. As the PolyhHb bracket decreases in size, the rate constant rapidly increases towards that of unmodified hHb (0.0995±0.0070 μM⁻¹ s⁻¹). All PolyhHb brackets exhibited significantly lower rate constants than unmodified hHb (p<0.05). High MW PolyhHbs, such as PolyhHb-B3 and PolyhHb-B4, are significantly slower than low MW PolyhHb, PolyhHb-B2 and PolyhHb-B1, when binding Hp (p<0.05). As the degree of cross-linking increases, Hp binding decreases in magnitude. The commercial polymerized HBOCs, PolyHeme® and Hemopure® possess Hp-binding rate constants of 0.003±0.000 and 0.010±0.001 μM⁻¹ s⁻¹ [40], respectively. These rate constants are consistent with that of PolyhHb-B2 (0.0323±0.0138 μM⁻¹ s⁻¹) and smaller MW PolyhHb brackets.

### Auto-Oxidation Kinetics

The rate at which hHb/PolyhHb was converted to metHb was measured using UV-visible spectrometry over 24 hours at 37° C. for all PolyhHb brackets and unmodified hHb. FIG. 5 shows the auto-oxidation kinetics for PolyhHb and hHb and Table 1 lists the rate constants. The PolyhHb brackets display biphasic auto-oxidation kinetics as indicated with a fast rate, k_{ox,fast}, followed by a slower rate, k_{ox,slow}. It has been speculated that these biphasic kinetics could be attributed to the difference in oxidation rates of the α chain versus the β chain[52]. The auto-oxidation of unmodified hHb is not biphasic within this time interval[53] and was fit to a single rate constant (0.0221±0.008 hr⁻¹). Both auto-oxidation trends are consistent with the literature[23,35]. There is a significant difference between the fast auto-oxidation rate constant of PolyhHb-B3 and the low MW PolyhHb brackets (p<0.05). There was no significant difference between the slow auto-oxidation rate constants of each PolyhHb bracket (p>0.05). When compared to hHb, there was a significant difference between hHb and slow rate constants of each PolyhHb bracket (p<0.05), but no significant difference between hHb and the fast rate constants (p>0.05). When measured in 50 mM PBS, pH 7.4, and 37° C. for 24 hr, the monophasic auto-oxidation kinetic rate constant of PolyHeme® and Hemopure® are 0.260±0.010 hr⁻¹ and 0.220±0.020 hr⁻¹, respectively[40]. The auto-oxidation rates of these commercial products are 10-fold faster than the native protein and ~20-fold faster than the PolyhHb brackets presented in this Example.

### Pharmacokinetics of PolyhHb Brackets in Exchange Transfused Guinea Pigs

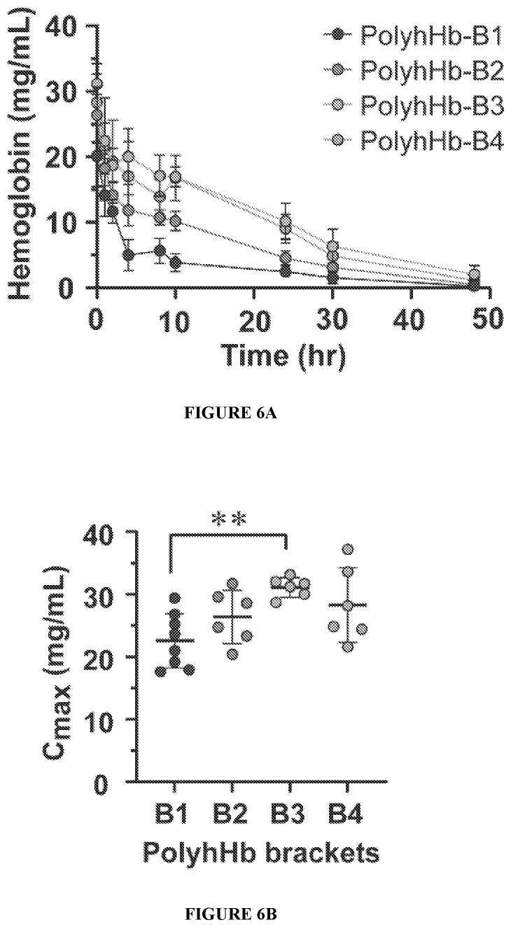

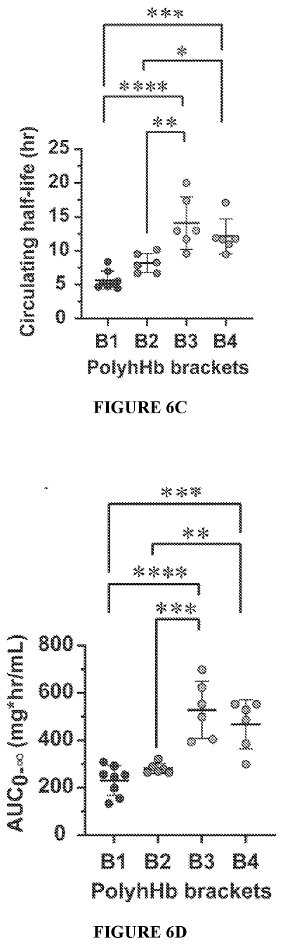

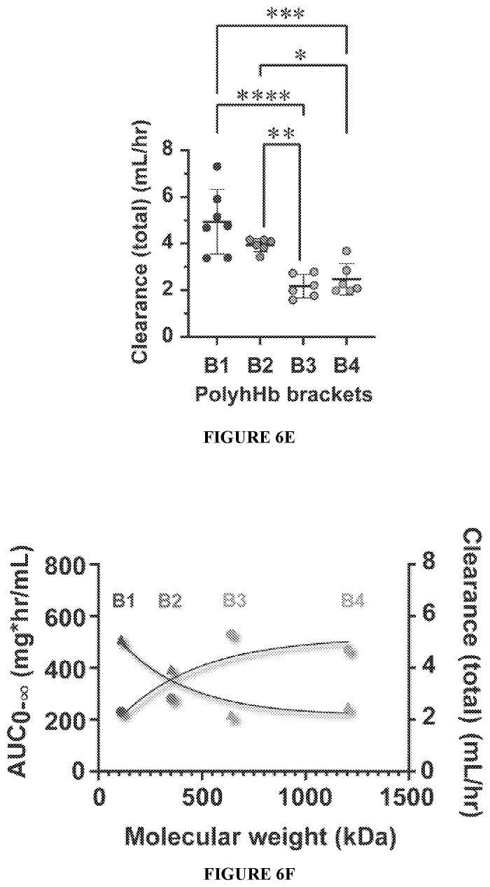

The PK behavior of chemically and recombinantly modified Hb is dependent on molecular size[22,36], stability[54], and physicochemical preferences for renal and non-renal clearance pathways[55,56]. We performed a 25% blood volume ET of PolyhHb (-B1, -B2, -B3 or -B4) and post ET time dependent blood collections. Plasma samples were analyzed using UV-visible spectrophotometric analysis. Non-compartmental analysis of plasma PolyhHb concentration versus time was performed and the data are shown in Table 2 for PK parameters. Selected PK parameters are plotted in FIG. 6A to allow for a visual comparison of exposure data and half-life. These data suggest a plateauing of PK parameters with PolyhHb MW greater than 586.2±100.3 kDa. The maximal PolyhHb concentrations in plasma (Cₘₐₓ) occurred immediately after ET as shown in FIG. 6B. Cₘₐₓ concentrations differed between PolyhHb-B1 and PolyhHb-B3 suggesting that clearance of PolyhHb-B1 is already occurring during the process of ET. The circulating half-life shown in FIG. 6C was derived from all points in the log linear plotted data and was found to increase from 5.66±1.34 hours after PolyhHb-B1 25% ET to 14.1±3.86 hours and 12.1±2.85 hours after 25% ET with PolyhHb-B3 and -B4, respectively. The overall exposures defined by area under the plasma concentration-time curve from the first blood sampling to infinity (AUC_{0-∞}) and the dose/AUC_{0-∞} derived total clearance is shown in FIGS. 6 D and E, respectively. AUC_{0-∞} values increased by 2-fold following 25% ET with both PolyhHb-B3 and PolyhHb-B4 compared to PolyhHb-B1, while the total body clearance decreased by the same magnitude. The saturation of PolyhHb AUC^{0-∞} and total body clearance in 25% ET guinea pigs are shown together in FIG. 6F as analyzed using the exponential plateau (for AUC_{0-∞}), and single-phase decay (for total clearance) function in GraphPad 9.1.1. These data suggest a saturation of PolyhHb PK parameters as bracketed material MW exceeds 586.2±100.3 kDa (i.e., bracket PolyhHb-B3). Interestingly, this data is consistent with the PK response to a 50% ET with polymerized bovine Hb prepared with glutaraldehyde:bovine Hb molar ratios of 10:1, 20:1, 30:1 or 40:1[26]. In these studies, polymerization ratio dependent PK parameters were not observed, and in fact circulation times decreased as the bovine Hb polymer MW exceeded 1 MDa[26]. Therefore, a MW of approximately 600 kDa appears to define maximal circulatory persistence and is consistent with the results in this Example.

TABLE 2
Pharmacokinetic parameter estimates following exchange transfusion.

| | PolyhHb-B1 | PolyhHb-B2 | PolyHb-B3 | PolyhHb-B4 |
|---|---|---|---|---|
| Parameters | [100 mg/mL] | [100 mg/mL] | [100 mg/mL] | 100 [mg/mL] |
| Dose (mg) | 1157 ± 116.9 | 1062 ± 223.7 | 1207 ± 120.0 | 1282 ± 132.1 |
| Cₘₐₓ (mg/mL) | 20.2 ± 4.71^{‡} | 26.4 ± 4.28 | 31.1 ± 1.56 | 28.3 ± 5.99 |
| T_{1/2} (hours) | 5.66 ± 1.34^{‡} | 6.9 ± 0.494^{‡} | 14.1 ± 3.86*^{†} | 12.1 ± 2.58*^{†} |
| AUCₗₐₛₜ (hours*mg/mL) | 136 ± 35.2^{‡} | 265 ± 22.4* | 495 ± 93.9*^{†} | 451 ± 103*^{†} |
| AUC_{0-∞} (hours*mg/mL) | 137 ± 35.0^{‡} | 266 ± 22.4* | 542 ± 134*^{†} | 468 ± 104*^{†} |
| Cl (mg/mL) | 8.50 ± 2.11^{‡} | 4.16 ± 0.324*^{†} | 2.14 ± 0.526*^{†} | 2.45 ± 0.668*^{†} |
| MRT (hours) | 11.6 ± 1.29^{‡} | 12.5 ± 0.915^{‡} | 19.7 ± 4.89*^{†} | 16.6 ± 2.21*^{†} |
| AUMC (hours*hours*mg/mL) | 1618 ± 519.4^{‡} | 3341 ± 378.5^{‡} | 11,218 ± 5549.6*^{†} | 7844 ± 2044*^{†} |
| V_{c} (mL) | 59.7 ± 11.7 | 41.8 ± 13.5* | 35.5 ± 1.82* | 40.3 ± 8.10* |

Cₘₐₓ: Maximum plasma concentration; T_{1/2}: Plasma half-life; AUCₗₐₛₜ: Area of the plasma concentration time curve to the last measurable PolyhHb concentration; AUC_{0-∞}: AUCₗₐₛₜ calculated to infinity using the log-linear trapezoidal rule; Cl: total body clearance (renal + non-renal); MRT; mean residence time; AUMC: Area under the first moment time curve; V_{c}; The central compartment volume of distribution. Measured parameters are reported as the mean ± standard deviation.
*Denotes a statistically significant difference (p < 0.05) compared to PolyhHb-1.
^{†}Denotes a statistically significant difference (p < 0.05) compared to LMW PolyhHb (PolyhHb-B1 and/or PolyhHb-B2).
^{‡}denotes a statistically significant difference (p < 0.05) compared to HMW PolyhHb (PolyhHb-B3 and/or PolyhHb-B4).

### Tissue Distribution and Metabolism of PolyhHb Brackets in Exchange Transfused Guinea Pigs

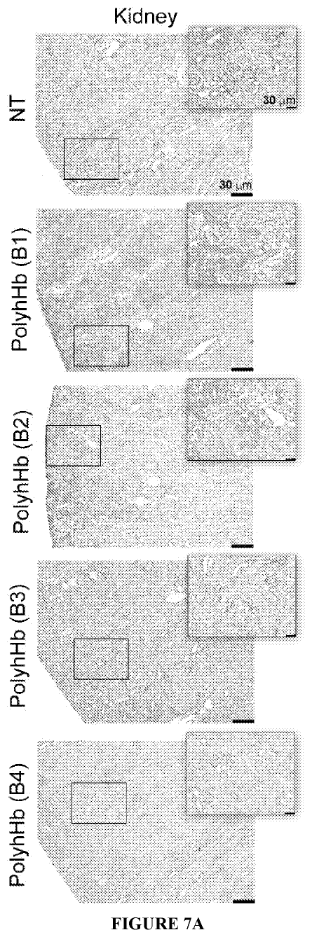

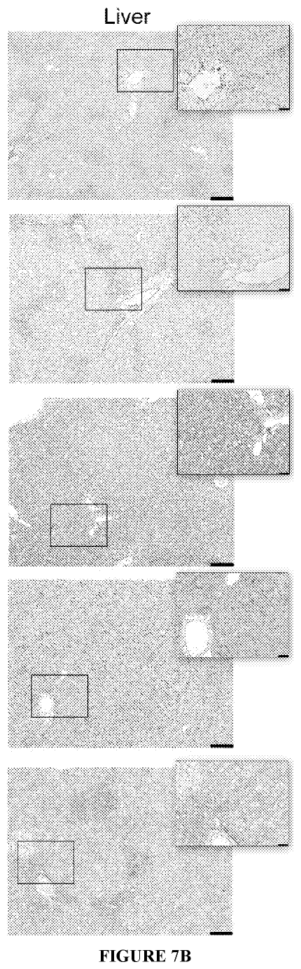

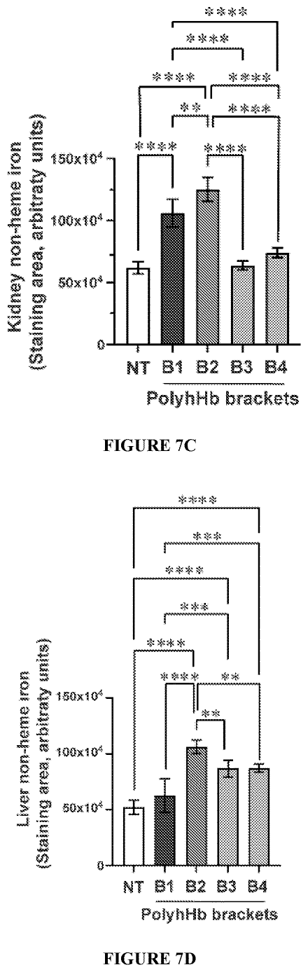

Recombinant and chemically modified Hb preparations distribute primarily to the kidneys and liver where they undergo filtration and metabolism[22,26,36]. In vivo, the distribution of modified Hb to tissues is a MW and charge dependent process[57]. Renal tissue distribution is based on glomerular filtration size limits[58], and determined by the dimerization or degradation of Hb tetramers[59,60]. Perls iron staining with DAB intensification of kidney and liver tissue sections was performed to understand the distribution of PolyhHb-B1 to -B4 as shown in FIGS. 7 A and B, respectively (both at 4× and 20× magnification). Based on animal studies, renal clearance of modified Hb is not desirable because of the potential for acute kidney injury with large volume transfusions[54]. As expected, the lower MW PolyhHb-B1 (64.7±0.2 kDa) and PolyhHb-B2 (105.8±5.8 kDa) fractions show the greatest visual iron staining in renal tubules, suggesting Hb dimerization. In contrast, PolyhHb-B3 and -B4 show no renal iron accumulation, consistent with Hp binding data shown in FIG. 4D. PolyhHb-B2 also shows a high visual distribution in the liver, indicating both renal and non-renal mechanisms of clearance that are unique to this bracketed material. The mean±SD quantitative image analysis demonstrates the PolyhHb group differences in kidney distribution shown in FIG. 7C and liver distribution shown in FIG. 7D.

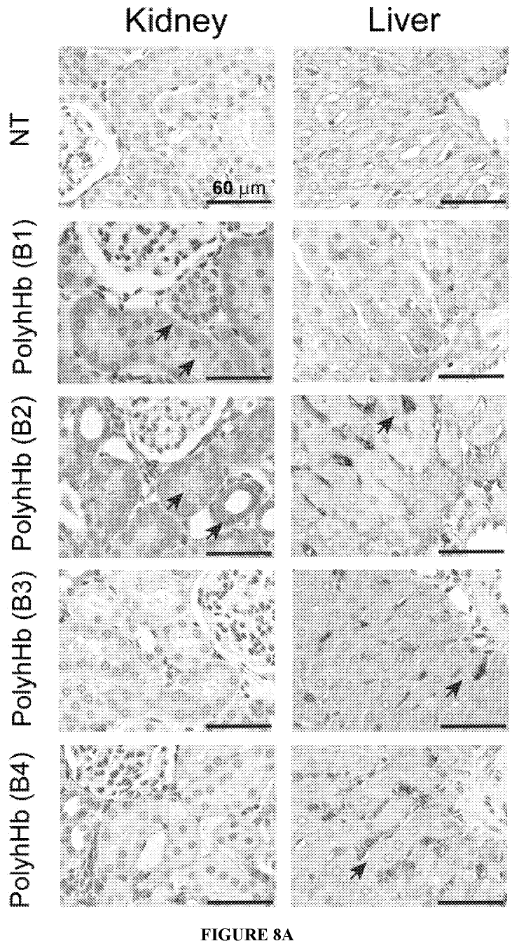

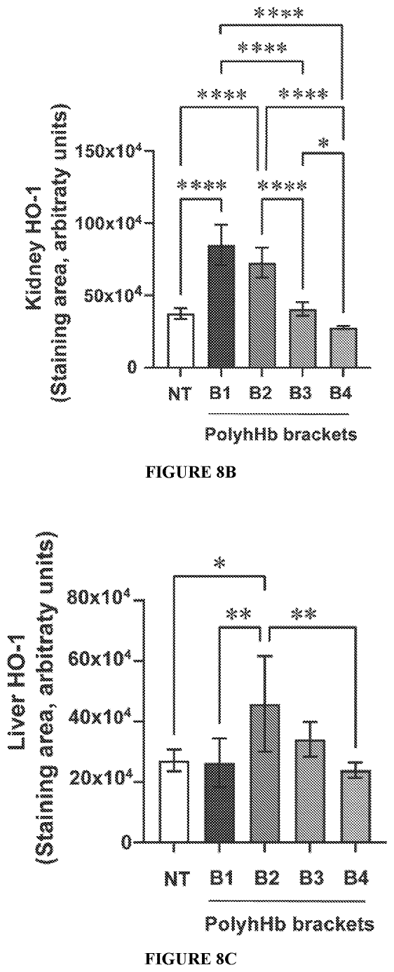

To understand the primary cellular locations of PolyhHb metabolism by heme oxygenase-1 (HO-1), we performed tissue immunohistochemistry (IHC) on sections prepared from the kidneys and liver. Immune reactivity for HO-1 in kidneys as shown in FIG. 8A is consistent with iron distribution to renal proximal and distal tubules. HO-1 expression in kidney proximal and distal tubules serve to protect these critical structures responsible for electrolyte and nutrient homeostasis[61]. However, large amounts of Hb excreted by glomerular filtration overcomes protective mechanisms leading to acute manifestations of kidney injury[62]. Both PolyhHb-B1 and -B2 show increased visualization of HO-1 expression in renal proximal and distal tubules, while PolyhHb-B3 and -B4 do not. These representative images are consistent with differences in group HO-1 quantitative data shown in FIG. 8B. Liver tissue sections from the PolyhHb-B2, -B3 and -B4 groups, but not the PolyhHb-B1 group show HO-1 expression of highest intensity in Kupffer cells (indicated by arrows). This observation is also consistent with the distribution of iron after ET with PolyhHb brackets. The group comparisons for HO-1 are consistent with differences in group quantitative data shown in FIG. 8C.

### Hemodynamic Measurements of PolyhHb Brackets in Exchange Transfused Guinea Pigs

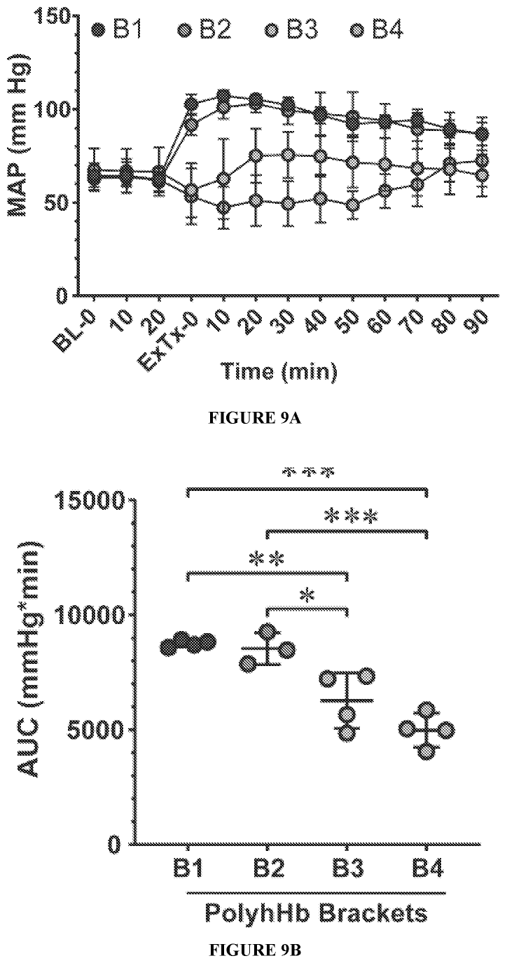

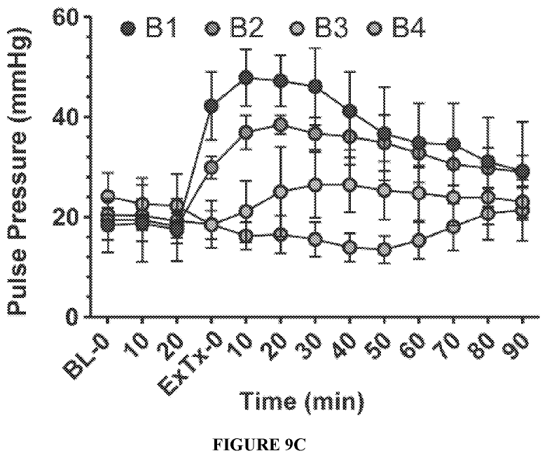

To determine the hemodynamic impact of the PolyhHb brackets (-B1 to -B4) in vivo, we performed a 25% exchange transfusion in guinea pigs (n=4 per bracket). Exchange transfusion of the lower MW PolyhHb brackets (-B1 and -B2) caused a higher MAP immediately following ET, while higher MW PolyhHb-B3 and -B4 maintained or slightly reduced MAP compared to baseline levels, respectively as shown in FIG. 9A. Over the 90-minutes post-transfusion, the MAP trended back towards the baseline pressure of approximately 65 mm Hg for PolyhHb brackets B1 and B2. To compare the total effect of each PolyhHb on blood pressure, the area under the curve (AUC) for each PolyhHb was calculated over the 90-minute interval. PolyhHb-B1 and -B2 led to significantly higher MAP AUCs when compared to PolyhHb-B3 and -B4 as shown in FIG. 9B. In addition to the impact on MAP, transfusion with PolyhHb brackets led to changes in pulse pressure (difference between systolic and diastolic pressures) that mirrored the MAP data, as shown in FIG. 9C. As with MAP, the pulse pressure trended towards baseline over the observation period.

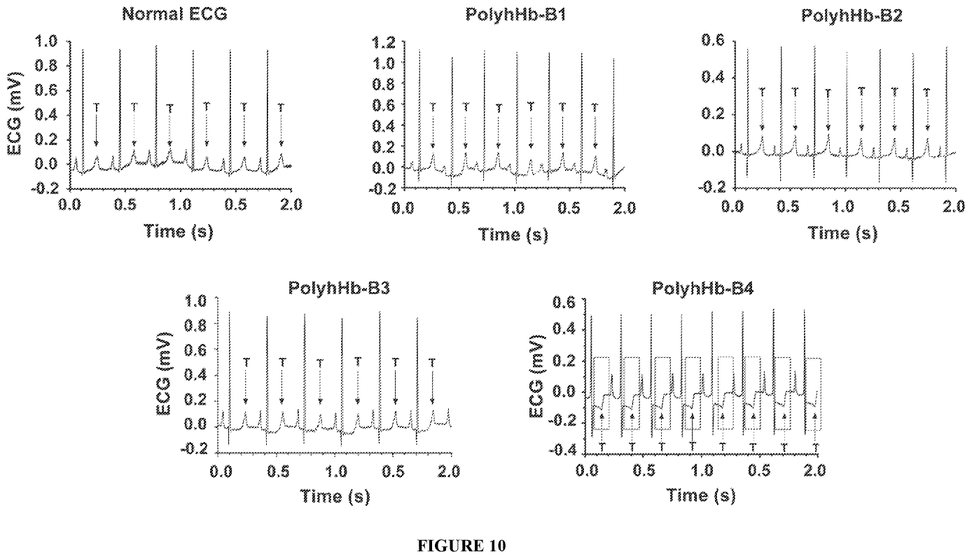

During the ET pressure experiments, we monitored guinea pigs' cardiac function using a lead ii ECG. Representative snapshots of these ECGs observed during 25% ET with each of the PolyhHb brackets are shown in FIG. 10. In three of the four animals dosed with the PolyhHb-B4 material, ECG readings showed repeated T wave inversion during the ET, an aberrant heart rhythm consistent with cardiac ischemia[63]. However, no abnormalities were observed in animals that received PolyhHb brackets (B1-B3). Taken together with extended circulatory pharmacokinetics, no renal tissue distribution, no aberrant blood pressure, or cardiac conduction effects, the PolyhHb-B3 bracket may offer the most appropriate material for further testing.

### Conclusion

This Example describes the production of PolyhHb with a range of glutaraldehyde polymerization ratios followed by TFF fractionation into four specific MW brackets (PolyhHb-B1 to -B4). Each of these brackets demonstrate unique biochemical and biophysical properties that suggest potential use as O₂ therapeutics in transfusion medicine. To further characterize their potential properties, we evaluated each PolyhHb in a 25% guinea pig model of exchange transfusion. This model is intended to screen based on PK parameters, tissue distribution, hemodynamics and cardiac ischemia (evaluated by ECG). Here we suggest that PolyhHb-B3 may offer an optimal preclinical screening profile to further study in a more advanced hybrid model to validate effectiveness and identify potential (exploratory) preclinical safety concerns.

### REFERENCES

[1]A. G. Greenburg, D. Ph, Benefits and Risks of Blood Transfusion in Surgical Patients, (1996) 1189-1193.
[2]C. P. Stowell, J. Levin, B. D. Spiess, R. M. Winslow, Progress in the Development of RBC substitutes, 41 (2001) 287-299.
[3]E. E. Moore, F. A. Moore, T. C. Fabian, A. C. Bernard, G. J. Fulda, D. B. Hoyt, T. M. Duane, L. J. Weireter, G. A. Gomez, M. D. Cipolle, G. H. Rodman, M. A. Malangoni, G. A. Hides, L. A. Omert, S. A. Gould, Human Polymerized Hemoglobin for the Treatment of Hemorrhagic Shock when Blood Is Unavailable: The USA Multicenter Trial, Journal of the American College of Surgeons. 208 (2009) 1-13. https://doi.org/10.1016/j.jamcollsurg.2008.09.023.
[4]E. Bucci, H. Kwansa, R. C. Koehler, B. Matheson, Development of Zero-Link Polymers of Hemoglobin, Which do not Extravasate and do not Induce Pressure Increases upon Infusion Enrico, Artificial Cells, Blood Substitutes, and Biotechnology. 35 (2008) 11-18.
[5]B. Q. H. Gibson, F. J. W. Roughton, The kinetics and equilibria of the reactions of nitric oxide with sheep haemoglobin, The Journal of Physiology. 136 (1956) 507-526.
[6]A. S. Kavdia, M., Tsoukias, N. M., & Popel, Model of nitric oxide diffusion in an arteriole: Impact of hemoglobin-based blood substitutes, American Journal of Physiology-Heart and Circulatory Physiology. 282 (2002) H2245-H2253.
[7]M. R. McCarthy, K. D. Vandegriff, R. M. Winslow, The role of facilitated diffusion in oxygen transport by cell-free hemoglobins: implications for the design of hemoglobin-based oxygen carriers, Biophysical Chemistry. 92 (2001) 103-117. https://doi.org/10.1016/S0301-4622(01)00194-6.
[8]R. M. Winslow, Red Cell Substitutes, Seminars in Hematology. 44 (2007) 51-59. https://doi.org/10.1053/J.SEMINHEMATOL.2006.09.013.
[9]M. Cullison, R. Mahon, G. McGwin, R. McCarron, R. Browning, C. Auker, Blood transfusions, blood storage, and correlation with elevated pulmonary arterial pressures, Transfusion. 59 (2019) 1259-1266. https://doi.org/10.1111/TRF.15122.
[10]O. I. Butt, P. W. Buehler, F. D'Agnillo, Blood-brain barrier disruption and oxidative stress in guinea pig after systemic exposure to modified cell-free hemoglobin, American Journal of Pathology. 178 (2011) 1316-1328. https://doi.org/10.1016/j.ajpath.2010.12.006.
[11]H. W. Kim, A. G. Greenburg, Artificial oxygen carriers as red blood cell substitutes: A selected review and current status, Artificial Organs. 28 (2004) 813-828. https://doi.org/10.1111/j.1525-1594.2004.07345.x.
[12]T. A. Silverman, R. B. Weiskopf, Hemoglobin-based oxygen carriers: current status and future directions, Transfusion. 49 (2009) 2495-2515. https://doi.org/doi:10.1111/j.1537-2995.2009.02356.x.
[13]D. Guillochon, M. W. Vijayalakshmi, A. Thiam-Sow, D. Thomas, A. Chevalier, Effect of glutaraldehyde on hemoglobin: functional aspects and Mossbauer parameters, Biochemistry and Cell Biology. 64 (1986) 29-37. https://doi.org/10.1139/o86-005.
[14]D. Guillochon, L. Esclade, M. H. Remy, D. Thomas, Studies on haemoglobin immobilized by cross-linking with glutaraldehyde Cross-linked soluble polymers and artificial membranes, Biochimica et Biophysica Acta (BBA)—Protein Structure. 670 (1981) 332-340. https://doi.org/10.1016/0005-2795(81)90105-7.
[15]A. I. Alayash, A. G. Summers, F. Wood, Y. Jia, Effects of Glutaraldehyde Polymerization on Oxygen Transport and Redox Properties of Bovine Hemoglobin, Archives of Biochemistry and Biophysics. 391 (2001) 225-234. https://doi.org/10.1006/ABBI.2001.2426.
[16]A. I. Alayash, Hemoglobin-based blood substitutes and the hazards of blood radicals Hemoglobin-based Blood Substitutes and the Hazards of Blood Radicals*, Free Radical Research. 33 (2000) 341-348. https://doi.org/10.1080/10715760000300881.
[17]T. M. S. Chang, Stablisation of enzymes by microencapsulation with a concentrated protein solution or by microencapsulation followed by cross-linking with glutaraldehyde, Biochemical and Biophysical Research Communications. 44 (1971) 1531-1536. https://doi.org/10.1016/S0006-291X(71)80260-7.
[18]T. M. S. Chang, L. Bulow, J. Jahr, H. Sakai, C. Yang, Nanobiotherapeutic Based Blood Substitutes, WORLD SCIENTIFIC, 2021. https://doi.org/10.1142/12054.
[19]J. Y. Chen, M. Scerbo, G. Kramer, A review of blood substitutes: Examining the history, clinical trial results, and ethics of hemoglobin-based oxygen carriers, Clinics. 64 (2009) 803-813. https://doi.org/10.1590/S1807-59322009000800016.
[20]N. Zhang, Y. Jia, G. Chen, P. Cabrales, A. F. Palmer, Biophysical Properties and Oxygenation Potential of High-Molecular-Weight Glutaraldehyde-Polymerized Human Hemoglobins Maintained in the Tense and Relaxed Quaternary States, Tissue Engineering Part A. 17 (2011) 927-940. https://doi.org/10.1089/ten.tea.2010.0353.
[21]D. R. Harris, A. F. Palmer, Modern Cross-linking Strategies for Synthesizing Acellular Hemoglobin-Based Oxygen Carriers, Biotechnology Progress. 24 (2008) 1215-1225. https://doi.org/10.1002/btpr.85.Modern.
[22]P. W. Buehler, Y. Zhou, P. Cabrales, Y. Jia, G. Sun, D. R. Harris, A. G. Tsai, M. Intaglietta, A. F. Palmer, Synthesis, biophysical properties and pharmacokinetics of ultrahigh molecular weight tense and relaxed state polymerized bovine hemoglobins, Biomaterials. 31 (2010) 3723-3735. https://doi.org/10.1016/J.BIOMATERIALS.2010.01.072.
[23]C. T. Cuddington, S. R. Wolfe, A. F. Palmer, Biophysical properties of tense quaternary state polymerized human hemoglobins bracketed between 500 kDa and 0.2 m in size, Biotechnology Progress. (2021) 1-8. https://doi.org/10.1002/btpr.3219.
[24]P. Cabrales, G. Sun, Y. Zhou, D. R. Harris, A. G. Tsai, M. Intaglietta, A. F. Palmer, Effects of the molecular mass of tense-state polymerized bovine hemoglobin on blood pressure and vasoconstriction, Journal of Applied Physiology. 107 (2009) 1548-1558. https://doi.org/10.1152/japplphysiol.00622.2009.
[25]P. Cabrales, Y. Zhou, D. R. Harris, A. F. Palmer, Tissue oxygenation after exchange transfusion with ultrahigh-molecular-weight tense- and relaxed-state polymerized bovine hemoglobins, American Journal of Physiology-Heart and Circulatory Physiology. 298 (2010) H1062-H1071. https://doi.org/10.1152/ajpheart.01022.2009.
[26]J. H. Baek, Y. Zhou, D. R. Harris, D. J. Schaer, A. F. Palmer, P. W. Buehler, Down Selection of Polymerized Bovine Hemoglobins for Use as Oxygen Releasing Therapeutics in a Guinea Pig Model, Toxicological Sciences. 127 (2012) 567-581. https://doi.org/10.1093/toxsci/kfs109.
[27]A. T. Williams, A. Lucas, C. R. Muller, C. Munoz, C. Bolden-Rush, A. F. Palmer, P. Cabrales, Resuscitation From Hemorrhagic Shock With Fresh and Stored Blood and Polymerized Hemoglobin, Shock (Augusta, Ga.). 54 (2020) 464-473. https://doi.org/10.1097/SHK.0000000000001530.
[28]D. A. Belcher, C. T. Cuddington, E. L. Martindale, I. S. Pires, A. F. Palmer, Controlled Polymerization and Ultrafiltration Increase the Consistency of Polymerized Hemoglobin for Use as an Oxygen Carrier, Bioconjugate Chemistry. 31 (2020) 605-621. https://doi.org/10.1021/acs.bioconjchem.9b00766.
[29]A. F. Palmer, G. Sun, D. R. Harris, Tangential flow filtration of hemoglobin, Biotechnology Progress. 25 (2009) 189-199. https://doi.org/i0.1002/btpr.119.
[30]A. F. Palmer, G. Sun, D. R. Harris, The quaternary structure of tetrameric hemoglobin regulates the oxygen affinity of polymerized hemoglobin, Biotechnol Prog. 25 (2009) 1803-1809. https://doi.org/10.1002/btpr.265.
[31]J. Drabkin, DL; Austin, Spectrophotometric studies II. Preparations from washed blood cells; nitric oxide hemoglobin and sulfhemoglobin, The Journal of Biological Chemistry. 112 (1935) 51-65. https://doi.org/0021-9258.
[32]Y. Zhou, Y. Jia, P. W. Buehler, G. Chen, P. Cabrales, A. F. Palmer, Synthesis, biophysical properties, and oxygenation potential of variable molecular weight glutaraldehyde-polymerized bovine hemoglobins with low and high oxygen affinity, Biotechnology Progress. 27 (2011) 1172-1184. https://doi.org/10.1002/BTPR.624.
[33]A. V. Hill, The Possible Effects of the Aggregation of the Molecules of Haemoglobin on its Dissociation Curve, The Journal of Physiology. 40 (1910) iv-vii.
[34]S. Rameez, A. F. Palmer, Simple method for preparing poly(ethylene glycol)-surface-conjugated liposome-encapsulated hemoglobins: Physicochemical properties, long-term storage stability, and their reactions with O2, CO, and NO, Langmuir. 27 (2011) 8829-8840. https://doi.org/10.1021/la201246m.
[35]X. Gu, C. Bolden-Rush, C. T. Cuddington, D. A. Belcher, C. Savla, I. S. Pires, A. F. Palmer, Comprehensive characterization of tense and relaxed quaternary state glutaraldehyde polymerized bovine hemoglobin as a function of cross-link density, Biotechnology and Bioengineering. 117 (2020) 2362-2376. https://doi.org/10.1002/BIT.27382.
[36]P. W. Buehler, F. D'Agnillo, V. Hoffman, A. I. Alayash, Effects of endogenous ascorbate on oxidation, oxygenation, and toxicokinetics of cell-free modified hemoglobin after exchange transfusion in rat and guinea pig., The Journal of Pharmacology and Experimental Therapeutics. 323 (2007) 49-60. https://doi.org/10.1124/jpet.107.126409.
[37]R. J. ANCILL, The blood volume of the normal guinea-pig., The Journal of Physiology. 132 (1956) 469-75. https://doi.org/10.1113/jphysiol.1956.sp005539.
[38]C. A. Schneider, W. S. Rasband, K. W. Eliceiri, NIH Image to ImageJ: 25 years of image analysis., Nature Methods. 9 (2012) 671-5. https://doi.org/10.1038/nmeth.2089.
[39]C. Cuddington, S. Moses, D. Belcher, N. Ramesh, I Andre Palmer, A. Palmer, Next-generation polymerized human hemoglobins in hepatic bioreactor simulations, (2020). https://doi.org/10.1002/btpr.2958.
[40]F. Meng, T. Kassa, S. Jana, F. Wood, X. Zhang, Y. Jia, F. D'Agnillo, A. I. Alayash, Comprehensive Biochemical and Biophysical Characterization of Hemoglobin-Based Oxygen Carrier Therapeutics: All HBOCs Are Not Created Equally, Bioconjugate Chemistry. 29 (2018) 1560-1575. https://doi.org/10.1021/acs.bioconjchem.8b00093.
[41]J. S. Jahr, M. Moallempour, J. C. Lim, HBOC-201, hemoglobin glutamer-250 (bovine), Hemopure® (Biopure Corporation), Expert Opinion on Biological Therapy. 8 (2008) 1425-1433. https://doi.org/10.1517/14712598.8.9.1425.
[42]S. A. Gould, E. E. Moore, D. B. Hoyt, J. M. Burch, J. B. Haenel, J. Garcia, R. DeWonskin, G. S. Moss, The first randomized trial of human polymerized hemoglobin as a blood substitute in acute trauma and emergent surgery, Journal of the American College of Surgeons. 187 (1998) 113-120. https://doi.org/10.1016/s1072-7515(98)00095-7.
[43]H. Sakai, H. Hara, M. Yuasa, A. G. Tsai, S. Takeoka, E. Tsuchida, M. Intaglietta, Molecular dimensions of Hb-based O2 carriers determine constriction of resistance arteries and hypertension, American Journal of Physiology—Heart and Circulatory Physiology. 279 (2000) 908-915. https://doi.org/10.1152/ajpheart.2000.279.3.h908.
[44]P. W. Buehler, F. D'Agnillo, D. J. Schaer, Hemoglobin-based oxygen carriers: from mechanisms of toxicity and clearance to rational drug design, Trends in Molecular Medicine. 16 (2010) 447-457. https://doi.org/doi:10.1016/j. molmed.2010.07.006.
[45]L. M. Napolitano, Hemoglobin-based Oxygen Carriers: First, Second or Third Generation? Human or Bovine? Where are we Now?, Critical Care Clinics. 25 (2009) 279-301. https://doi.org/10.1016/j.ccc.2009.01.003.
[46]×. Gu, C. Savla, A. F. Palmer, Tangential Flow Filtration Facilitated Fractionation and PEGylation of Low and High Molecular Weight Polymerized Hemoglobins and Their Biophysical Properties, Biotechnology and Bioengineering. (2021). https://doi.org/10.1002/bit.27962.
[47]S. A. Gould, G. S. Moss, Clinical development of human polymerized hemoglobin as a blood substitute, World Journal of Surgery. 20 (1996) 1200-1207. https://doi.org/10.1007/s002689900183.
[48]T. K. Day, Current development and use of hemoglobin-based oxygen-carrying (HBOC) solutions, Journal of Veterinary Emergency and Critical Care. 13 (2003) 77-93. https://doi.org/10.1046/j.1435-6935.2003.00084.x.
[49]A. F. Palmer, N. Zhang, Y. Zhou, D. R. Harris, P. Cabrales, Small-Volume resuscitation from hemorrhagic shock using high-molecular wight tense-state polymerized hemoglobins, The Journal of Trauma: Injury, Infection, and Critical Care. 71 (2011) 798-807. https://doi.org/10.1097/TA.0b013e3182028ab0.
[50]M. Skiba, M. Skiba-Lahiani, P. Arnaud, Design of nanocapsules based on novel fluorophilic cyclodextrin derivatives and their potential role in oxygen delivery, in: Journal of Inclusion Phenomena, 2002: pp. 151-154. https://doi.org/10.1023/A:1023090528809.
[51]A. W. Y. Shih, A. Mcfarlane, M. Verhovsek, Haptoglobin testing in hemolysis: Measurement and interpretation, (n.d.). https://doi.org/10.1002/ajh.23623.
[52]M. Tsuruga, A. Matsuoka, A. Hachimori, Y. Sugawara, K. Shikama, The molecular mechanism of autoxidation for human oxyhemoglobin. Tilting of the distal histidine causes nonequivalent oxidation in the R chain, Journal of Biological Chemistry. 273 (1998) 8607-8615. https://doi.org/10.1074/jbc.273.15.8607.
[53]M. Tsuruga, K. Shikama, Biphasic nature in the autoxidation reaction of human oxyhemoglobin, Biochimica et Biophysica Acta (BBA)—Protein Structure and Molecular Enzymology. 1337 (1997) 96-104. https://doi.org/10.1016/S0167-4838(96)00156-2.
[54]J. H. Baek, A. Yalamanoglu, R. P. Brown, D. M. Saylor, R. A. Malinauskas, P. W. Buehler, Renal Toxicodynamic Effects of Extracellular Hemoglobin After Acute Exposure., Toxicological Sciences: An Official Journal of the Society of Toxicology. 166 (2018) 180-191. https://doi.org/10.1093/toxsci/kfyl93.
[55]P. W. Buehler, F. Vallelian, M. G. Mikolajczyk, G. Schoedon, T. Schweizer, A. I. Alayash, D. J. Schaer, Structural stabilization in tetrameric or polymeric hemoglobin determines its interaction with endogenous antioxidant scavenger pathways., Antioxidants & Redox Signaling. 10 (2008) 1449-62. https://doi.org/10.1089/ars.2008.2028.
[56]F. S. Boretti, J. H. Baek, A. F. Palmer, D. J. Schaer, P. W. Buehler, Modeling hemoglobin and hemoglobin:haptoglobin complex clearance in a non-rodent species-pharmacokinetic and therapeutic implications., Frontiers in Physiology. 5 (2014) 385. https://doi.org/10.3389/fphys.2014.00385.
[57]K. Taguchi, S. Nagao, K. Yamasaki, H. Sakai, H. Seo, T. Maruyama, M. Otagiri, Biological Responsiveness and Metabolic Performance of Liposome-Encapsulated Hemoglobin (Hemoglobin-Vesicles) in Apolipoprotein E-Deficient Mice after Massive Intravenous Injection., Biological & Pharmaceutical Bulletin. 38 (2015) 1606-16. https://doi.org/10.1248/bpb.b15-00420.
[58]B. Haraldsson, J. Nyström, W. M. Deen, Properties of the glomerular barrier and mechanisms of proteinuria., Physiological Reviews. 88 (2008) 451-87. https://doi.org/10.1152/physrev.00055.2006.
[59]H. F. Bunn, W. T. Esham, R. W. Bull, The renal handling of hemoglobin. I. Glomerular filtration., The Journal of Experimental Medicine. 129 (1969) 909-23. https://doi.org/10.1084/jem.129.5.909.
[60]H. F. Bunn, J. H. Jandl, The renal handling of hemoglobin., Transactions of the Association of American Physicians. 81 (1968) 147-52.
[61]A. Grunenwald, L. T. Roumenina, M. Frimat, Heme Oxygenase 1: A Defensive Mediator in Kidney Diseases., International Journal of Molecular Sciences. 22 (2021). https://doi.org/10.3390/ijms22042009.
[62]J. Hu, E. Rezoagli, F. Zadek, E. A. Bittner, C. Lei, L. Berra, Free Hemoglobin Ratio as a Novel Biomarker of Acute Kidney Injury After On-Pump Cardiac Surgery: Secondary Analysis of a Randomized Controlled Trial., Anesthesia and Analgesia. 132 (2021) 1548-1558. https://doi.org/10.1213/ANE.0000000000005381.
[63]K. Channer, F. Morris, ABC of clinical electrocardiography: Myocardial ischaemia., BMJ (Clinical Research Ed.). 324 (2002) 1023-6. https://doi.org/10.1136/bmj.324.7344.1023.

Other advantages which are obvious, and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

What is claimed is:

1. A hemoglobin-based oxygen carrier (HBOC) composition comprising a polymerized hemoglobin (PolyHb),
wherein the composition comprises less than 10% by weight hemoglobin species having a molecular weight of less than 500 kDa, based on the total weight of the HBOC composition; and
wherein the composition comprises less than 10% by weight hemoglobin species having a molecular weight of greater than 750 kDa, based on the total weight of the HBOC composition.

2. The composition of claim 1, wherein the composition comprises less than 5% by weight hemoglobin species having a molecular weight of less than 500 kDa, based on the total weight of the HBOC composition, such as less than 2.5% by weight hemoglobin species having a molecular weight of less than 500 kDa, less than 1% by weight hemoglobin species having a molecular weight of less than 500 kDa, less than 0.5% by weight hemoglobin species having a molecular weight of less than 500 kDa, less than 0.1% by weight hemoglobin species having a molecular weight of less than 500 kDa, or less than 0.05% by weight hemoglobin species having a molecular weight of less than 500 kDa.

3. The composition of any of claims 1-2, wherein the composition comprises less than 5% by weight hemoglobin species having a molecular weight of greater than 750 kDa, based on the total weight of the HBOC composition, such as less than 2.5% by weight hemoglobin species having a molecular weight of greater than 750 kDa, less than 1% by weight hemoglobin species having a molecular weight of greater than 750 kDa, less than 0.5% by weight hemoglobin species having a molecular weight of greater than 750 kDa, less than 0.1% by weight hemoglobin species having a molecular weight of greater than 750 kDa, or less than 0.05% by weight hemoglobin species having a molecular weight of greater than 750 kDa.

4. The composition of any of claims 1-3, wherein the composition comprises less than 10% by weight hemoglobin species having a molecular weight of less than 300 kDa, based on the total weight of the HBOC composition, such as less than 5% by weight hemoglobin species having a molecular weight of less than 300 kDa, less than 2.5% by weight hemoglobin species having a molecular weight of less than 300 kDa, less than 1% by weight hemoglobin species having a molecular weight of less than 300 kDa, less than 0.5% by weight hemoglobin species having a molecular weight of less than 300 kDa, less than 0.1% by weight hemoglobin species having a molecular weight of less than 300 kDa, or less than 0.05% by weight hemoglobin species having a molecular weight of less than 300 kDa.

5. The composition of any of claims 1-4, wherein the composition comprises less than 10% by weight hemoglobin species having a hydrodynamic diameter of greater than 50 nm, based on the total weight of the HBOC composition, such as less than 5% by weight hemoglobin species having a hydrodynamic diameter of greater than 50 nm, less than 2.5% by weight hemoglobin species having a hydrodynamic diameter of greater than 50 nm, less than 1% by weight hemoglobin species having a hydrodynamic diameter of greater than 50 nm, less than 0.5% by weight hemoglobin species having a hydrodynamic diameter of greater than 50 nm, less than 0.1% by weight hemoglobin species having a hydrodynamic diameter of greater than 50 nm, or less than 0.05% by weight hemoglobin species having a hydrodynamic diameter of greater than 50 nm.

6. The composition of any of claims 1-5, wherein the composition comprises less than 10% by weight hemoglobin species having a hydrodynamic diameter of greater than 0.1 microns, based on the total weight of the HBOC composition, such as less than 5% by weight hemoglobin species having a hydrodynamic diameter of greater than 0.1 microns, less than 2.5% by weight hemoglobin species having a hydrodynamic diameter of greater than 0.1 microns, less than 1% by weight hemoglobin species having a hydrodynamic diameter of greater than 0.1 microns, less than 0.5% by weight hemoglobin species having a hydrodynamic diameter of greater than 0.1 microns, less than 0.1% by weight hemoglobin species having a hydrodynamic diameter of greater than 0.1 microns, or less than 0.05% by weight hemoglobin species having a hydrodynamic diameter of greater than 0.1 microns.

7. The composition of any of claims 1-6, wherein a majority of hemoglobin (Hb) in the PolyHb is locked in at least one of a tense quaternary state or a relaxed quaternary state.

8. The composition of any of claims 1-7, wherein a majority of hemoglobin (Hb) in the PolyHb is locked in a tense quaternary state.

9. The composition of any of claims 1-7, wherein a majority of hemoglobin (Hb) in the PolyHb is locked in a relaxed quaternary state.

10. The composition of any of claims 1-9, wherein the PolyHb has a cross-linker to Hb molar ratio of at least about 20:1, such as at least about 25:1.

11. The composition of any of claims 1-10, wherein the PolyHb has a cross-linker to Hb molar ratio of from about 25:1 to about 30:1.

12. The composition of any of claims 1-11, wherein the PolyHb is polymerized using a cross-linker comprising glutaraldehyde, succindialdehyde, activated forms of polyoxyethylene and dextran, -hydroxy aldehydes, such as glycolaldehyde, N-maleimido-6-aminocaproyl-(2′-nitro,4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylarninopropyl)carbodiimide hydrochloride, N,N′-phenylene dimaleimide, or a combination thereof.

13. The composition of any of claims 1-12, wherein the PolyHb is polymerized using a dialdehyde crosslinker.

14. The composition of any of claims 1-13, wherein the PolyHb is polymerized using glutaraldehyde.

15. The composition of any of claims 1-14, wherein the polymerized hemoglobin is prepared by a process that comprises:
polymerizing hemoglobin to form a precursor solution;
filtering the precursor solution by ultrafiltration against a first filtration membrane having a pore size that separates hemoglobin species having a molecular weight of less than 500 kDa from the polymerized hemoglobin; and
filtering the precursor solution by ultrafiltration against a second filtration membrane having a pore size that separates hemoglobin species having a molecular weight of greater than 750 kDa from the polymerized hemoglobin.

16. The composition of claim 15, wherein the ultrafiltration comprises tangential-flow filtration.

17. The composition of any of claims 15-16, wherein polymerizing hemoglobin comprises:
adjusting the pO₂ of a solution containing Hb to a desired level,
polymerizing the Hb with a cross-linker while maintaining the desired pO₂, and collecting the polymerized hemoglobin in the precursor solution.

18. The composition of claim 17, wherein the desired level is the pO₂ wherein between 0% and about 10% of the Hb is saturated with O₂.

19. The composition of claim 17, wherein the desired level is the pO₂ wherein between about 90% and 100% of the Hb is saturated with O₂.

20. The composition of any of claims 17-19, wherein the pO₂ adjusting step includes any gas liquid exchange technology.

21. The composition of any of claims 17-20, wherein the pO₂ adjusting step further includes the addition of an oxygen scavenging agent.

22. A hemoglobin-based oxygen carrier (HBOC) composition comprising a polymerized hemoglobin (PolyHb),
wherein the composition comprises less than 10% by weight hemoglobin species having a molecular weight of less than 500 kDa, based on the total weight of the HBOC composition; and
wherein the composition comprises less than 10% by weight hemoglobin species having a hydrodynamic diameter of greater than 50 nm, based on the total weight of the HBOC composition.

23. The composition of claim 22, wherein the composition comprises less than 5% by weight hemoglobin species having a molecular weight of less than 500 kDa, based on the total weight of the HBOC composition, such as less than 2.5% by weight hemoglobin species having a molecular weight of less than 500 kDa, less than 1% by weight hemoglobin species having a molecular weight of less than 500 kDa, less than 0.5% by weight hemoglobin species having a molecular weight of less than 500 kDa, less than 0.1% by weight hemoglobin species having a molecular weight of less than 500 kDa or less than 0.05% by weight hemoglobin species having a molecular weight of less than 500 kDa.

24. The composition of any of claims 22-23, wherein the composition comprises less than 10% by weight hemoglobin species having a molecular weight of less than 300 kDa, based on the total weight of the HBOC composition, such as less than 5% by weight hemoglobin species having a molecular weight of less than 300 kDa, less than 2.5% by weight hemoglobin species having a molecular weight of less than 300 kDa, less than 1% by weight hemoglobin species having a molecular weight of less than 300 kDa, less than 0.5% by weight hemoglobin species having a molecular weight of less than 300 kDa, less than 0.1% by weight hemoglobin species having a molecular weight of less than 300 kDa, or less than 0.05% by weight hemoglobin species having a molecular weight of less than 300 kDa.

25. The composition of any of claims 22-24, wherein the composition comprises less than 10% by weight hemoglobin species having a molecular weight of greater than 750 kDa, based on the total weight of the HBOC composition, such as less than 5% by weight hemoglobin species having a molecular weight of greater than 750 kDa, less than 2.5% by weight hemoglobin species having a molecular weight of greater than 750 kDa, less than 1% by weight hemoglobin species having a molecular weight of greater than 750 kDa, less than 0.5% by weight hemoglobin species having a molecular weight of greater than 750 kDa, less than 0.1% by weight hemoglobin species having a molecular weight of greater than 750 kDa, or less than 0.05% by weight hemoglobin species having a molecular weight of greater than 750 kDa.

26. The composition of any of claims 22-25, wherein the composition comprises less than 10% by weight hemoglobin species having a hydrodynamic diameter of greater than 50 nm, based on the total weight of the HBOC composition, such as less than 5% by weight hemoglobin species having a hydrodynamic diameter of greater than 50 nm, less than 2.5% by weight hemoglobin species having a hydrodynamic diameter of greater than 50 nm, less than 1% by weight hemoglobin species having a hydrodynamic diameter of greater than 50 nm, less than 0.5% by weight hemoglobin species having a hydrodynamic diameter of greater than 50 nm, less than 0.1% by weight hemoglobin species having a hydrodynamic diameter of greater than 50 nm, or less than 0.05% by weight hemoglobin species having a hydrodynamic diameter of greater than 50 nm.

27. The composition of any of claims 22-26, wherein the composition comprises less than 10% by weight hemoglobin species having a hydrodynamic diameter of greater than 0.1 microns, based on the total weight of the HBOC composition, such as less than 5% by weight hemoglobin species having a hydrodynamic diameter of greater than 0.1 microns, less than 2.5% by weight hemoglobin species having a hydrodynamic diameter of greater than 0.1 microns, less than 1% by weight hemoglobin species having a hydrodynamic diameter of greater than 0.1 microns, less than 0.5% by weight hemoglobin species having a hydrodynamic diameter of greater than 0.1 microns, less than 0.1% by weight hemoglobin species having a hydrodynamic diameter of greater than 0.1 microns, or less than 0.05% by weight hemoglobin species having a hydrodynamic diameter of greater than 0.1 microns.

28. The composition of any of claims 22-27, wherein a majority of hemoglobin (Hb) in the PolyHb is locked in at least one of a tense quaternary state or a relaxed quaternary state.

29. The composition of any of claims 22-28, wherein a majority of hemoglobin (Hb) in the PolyHb is locked in a tense quaternary state.

30. The composition of any of claims 22-28, wherein a majority of hemoglobin (Hb) in the PolyHb is locked in a relaxed quaternary state.

31. The composition of any of claims 22-30, wherein the PolyHb has a cross-linker to Hb molar ratio of at least about 20:1, such as at least about 25:1.

32. The composition of any of claims 22-31, wherein the PolyHb has a cross-linker to Hb molar ratio of from about 25:1 to about 30:1.

33. The composition of any of claims 22-32, wherein the PolyHb is polymerized using a cross-linker comprising glutaraldehyde, succindialdehyde, activated forms of polyoxyethylene and dextran, -hydroxy aldehydes, such as glycolaldehyde, N-maleimido-6-aminocaproyl-(2′-nitro,4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylarninopropyl)carbodiimide hydrochloride, N,N′-phenylene dimaleimide, or a combination thereof.

34. The composition of any of claims 22-33, wherein the PolyHb is polymerized using a dialdehyde crosslinker.

35. The composition of any of claims 22-34, wherein the PolyHb is polymerized using glutaraldehyde.

36. The composition of any of claims 22-35, wherein the polymerized hemoglobin is prepared by a process that comprises:
polymerizing hemoglobin to form a precursor solution;
filtering the precursor solution by ultrafiltration against a first filtration membrane having a pore size that separates hemoglobin species having a molecular weight of less than 500 kDa from the polymerized hemoglobin; and
filtering the precursor solution by ultrafiltration against a second filtration membrane having a pore size that separates hemoglobin species having a hydrodynamic diameter of greater than 50 nm from the polymerized hemoglobin.

37. The composition of claim 36, wherein the ultrafiltration comprises tangential-flow filtration.

38. The composition of any of claims 36-37, wherein polymerizing hemoglobin comprises:
adjusting the pO₂ of a solution containing Hb to a desired level,
polymerizing the Hb with a cross-linker while maintaining the desired pO₂, and
collecting the polymerized hemoglobin in the precursor solution.

39. The composition of claim 38, wherein the desired level is the pO₂ wherein between 0% and about 10% of the Hb is saturated with O₂.

40. The composition of claim 38, wherein the desired level is the pO₂ wherein between about 90% and 100% of the Hb is saturated with O₂.

41. The composition of any of claims 38-40, wherein the pO₂ adjusting step includes any gas liquid exchange technology.

42. The composition of any of claims 38-41, wherein the pO₂ adjusting step further includes the addition of an oxygen scavenging agent.

43. A hemoglobin-based oxygen carrier (HBOC) composition comprising a polymerized hemoglobin (PolyHb),
wherein the composition comprises less than 10% by weight hemoglobin species having a molecular weight of less than 500 kDa, based on the total weight of the HBOC composition; and
wherein the composition comprises less than 10% by weight hemoglobin species having a hydrodynamic diameter of greater than 0.1 microns, based on the total weight of the HBOC composition.

44. The composition of claim 43, wherein the composition comprises less than 10% by weight hemoglobin species having a molecular weight of less than 500 kDa, based on the total weight of the HBOC composition, such as less than 5% by weight hemoglobin species having a molecular weight of less than 500 kDa, less than 2.5% by weight hemoglobin species having a molecular weight of less than 500 kDa, less than 1% by weight hemoglobin species having a molecular weight of less than 500 kDa, less than 0.5% by weight hemoglobin species having a molecular weight of less than 500 kDa, less than 0.1% by weight hemoglobin species having a molecular weight of less than 500 kDa or less than 0.05% by weight hemoglobin species having a molecular weight of less than 500 kDa.

45. The composition of any of claims 43-44, wherein the composition comprises less than 10% by weight hemoglobin species having a molecular weight of less than 300 kDa, based on the total weight of the HBOC composition, such as less than 5% by weight hemoglobin species having a molecular weight of less than 300 kDa, less than 2.5% by weight hemoglobin species having a molecular weight of less than 300 kDa, less than 1% by weight hemoglobin species having a molecular weight of less than 300 kDa, less than 0.5% by weight hemoglobin species having a molecular weight of less than 300 kDa, less than 0.1% by weight hemoglobin species having a molecular weight of less than 300 kDa, or less than 0.05% by weight hemoglobin species having a molecular weight of less than 300 kDa.

46. The composition of any of claims 43-45, wherein the composition comprises less than 10% by weight hemoglobin species having a molecular weight of greater than 750 kDa, based on the total weight of the HBOC composition, such as less than 5% by weight hemoglobin species having a molecular weight of greater than 750 kDa, less than 2.5% by weight hemoglobin species having a molecular weight of greater than 750 kDa, less than 1% by weight hemoglobin species having a molecular weight of greater than 750 kDa, less than 0.5% by weight hemoglobin species having a molecular weight of greater than 750 kDa, less than 0.1% by weight hemoglobin species having a molecular weight of greater than 750 kDa, or less than 0.05% by weight hemoglobin species having a molecular weight of greater than 750 kDa.

47. The composition of any of claims 43-46, wherein the composition comprises less than 10% by weight hemoglobin species having a hydrodynamic diameter of greater than 50 nm, based on the total weight of the HBOC composition, such as less than 5% by weight hemoglobin species having a hydrodynamic diameter of greater than 50 nm, less than 2.5% by weight hemoglobin species having a hydrodynamic diameter of greater than 50 nm, less than 1% by weight hemoglobin species having a hydrodynamic diameter of greater than 50 nm, less than 0.5% by weight hemoglobin species having a hydrodynamic diameter of greater than 50 nm, less than 0.1% by weight hemoglobin species having a hydrodynamic diameter of greater than 50 nm, or less than 0.05% by weight hemoglobin species having a hydrodynamic diameter of greater than 50 nm.

48. The composition of any of claims 43-47, wherein the composition comprises less than 10% by weight hemoglobin species having a hydrodynamic diameter of greater than 0.1 microns, based on the total weight of the HBOC composition, such as less than 5% by weight hemoglobin species having a hydrodynamic diameter of greater than 0.1 microns, less than 2.5% by weight hemoglobin species having a hydrodynamic diameter of greater than 0.1 microns, less than 1% by weight hemoglobin species having a hydrodynamic diameter of greater than 0.1 microns, less than 0.5% by weight hemoglobin species having a hydrodynamic diameter of greater than 0.1 microns, less than 0.1% by weight hemoglobin species having a hydrodynamic diameter of greater than 0.1 microns, or less than 0.05% by weight hemoglobin species having a hydrodynamic diameter of greater than 0.1 microns.

49. The composition of any of claims 43-48, wherein a majority of hemoglobin (Hb) in the PolyHb is locked in at least one of a tense quaternary state or a relaxed quaternary state.

50. The composition of any of claims 43-49, wherein a majority of hemoglobin (Hb) in the PolyHb is locked in a tense quaternary state.

51. The composition of any of claims 43-49, wherein a majority of hemoglobin (Hb) in the PolyHb is locked in a relaxed quaternary state.

52. The composition of any of claims 43-51, wherein the PolyHb has a cross-linker to Hb molar ratio of at least about 20:1, such as at least about 25:1.

53. The composition of any of claims 43-52, wherein the PolyHb has a cross-linker to Hb molar ratio of from about 25:1 to about 30:1.

54. The composition of any of claims 43-53, wherein the PolyHb is polymerized using a cross-linker comprising glutaraldehyde, succindialdehyde, activated forms of polyoxyethylene and dextran, -hydroxy aldehydes, such as glycolaldehyde, N-maleimido-6-aminocaproyl-(2′-nitro,4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylarninopropyl)carbodiimide hydrochloride, N,N′-phenylene dimaleimide, or a combination thereof.

55. The composition of any of claims 43-54, wherein the PolyHb is polymerized using a dialdehyde crosslinker.

56. The composition of any of claims 43-55, wherein the PolyHb is polymerized using glutaraldehyde.

57. The composition of any of claims 43-56, wherein the polymerized hemoglobin is prepared by a process that comprises:
polymerizing hemoglobin to form a precursor solution;
filtering the precursor solution by ultrafiltration against a first filtration membrane having a pore size that separates hemoglobin species having a molecular weight of less than 500 kDa from the polymerized hemoglobin; and
filtering the precursor solution by ultrafiltration against a second filtration membrane having a pore size that separates hemoglobin species having a hydrodynamic diameter of greater than 0.1 microns from the polymerized hemoglobin.

58. The composition of claim 57, wherein the ultrafiltration comprises tangential-flow filtration.

59. The composition of any of claims 57-58, wherein polymerizing hemoglobin comprises:
adjusting the pO₂ of a solution containing Hb to a desired level,
polymerizing the Hb with a cross-linker while maintaining the desired pO₂, and
collecting the polymerized hemoglobin in the precursor solution.

60. The composition of claim 59, wherein the desired level is the pO₂ wherein between 0% and about 10% of the Hb is saturated with O₂.

61. The composition of claim 59, wherein the desired level is the pO₂ wherein between about 90% and 100% of the Hb is saturated with O₂.

62. The composition of any of claims 59-61, wherein the pO₂ adjusting step includes any gas liquid exchange technology.

63. The composition of any of claims 59-62, wherein the pO₂ adjusting step further includes the addition of an oxygen scavenging agent.

64. The composition of any of claims 1-63, wherein the HBOC composition comprises 0.5-20 g/dL of the polymerized hemoglobin, 25-85 mM NaCl, 1-3 mM KCl, 6-20 mM KH₂PO₄, 20-70 mM sodium gluconate, 5-21 mM sodium lactate, 1-4 mM magnesium gluconate, 0.6-1.2 mM CaCl₂ dihydrate, 11-16 mM NaOH, 1-4 mM adenine, 2-8 mM dextrose, 0.5-3 mM glutathione, 2-8 mM HEPES, 1-4 mM ribose, 7-30 mM mannitol, 10-40 g/L hydroxyethyl starch, and 40-160 mg/dL N-acetyl-L-cysteine.

65. The perfusion solution of any of claims 1-64, wherein the HBOC composition comprises 3-4 g/dL the polymerized hemoglobin, 25-85 mM NaCl, 1-3 mM KCl, 6-20 mM KH₂PO₄, 20-70 mM sodium gluconate, 5-21 mM sodium lactate, 1-4 mM magnesium gluconate, 0.6-1.2 mM CaCl₂ dihydrate, 11-16 mM NaOH, 1-4 mM adenine, 2-8 mM dextrose, 0.5-3 mM glutathione, 2-8 mM HEPES, 1-4 mM ribose, 7-30 mM mannitol, 10-40 g/L hydroxyethyl starch, and 40-160 mg/dL N-acetyl-L-cysteine.

66. The composition of any of claims 1-65, wherein the HBOC composition comprises from 1% to 5% by weight albumin, based on total weight of the HBOC composition.

67. The composition of any of claims 1-66, wherein the HBOC composition has an osmolarity from 270 to 370 mOsm, such as an osmolarity from 324 to 346 mOsm.

68. The composition of any of claims 1-67, wherein the HBOC composition has a viscosity from 1 cP to 10 cP at physiological temperature, such as a viscosity from 2 to 4 cP at physiological temperature.

69. The composition of any of claims 1-68, wherein the HBOC composition has a colloid osmotic pressure from 1 mm Hg to 60 mm Hg, such as a colloid osmotic pressure from 10 to 30 mm Hg.

70. The composition of any of claims 1-69, wherein the partial pressure of oxygen at which 50% of the polymerized hemoglobin is saturated with oxygen is from 1 mm Hg to 60 mm Hg, such as from 20 mm Hg to 50 mm Hg.

71. The composition of any of claims 1-70, wherein the polymerized hemoglobin exhibits an auto-oxidation rate constant at 37° C. from 0.0020 to 0.05 h⁻¹, such as from 0.0025 to 0.05 h⁻¹.

72. The composition of any of claim 1-71, wherein the HBOC composition further comprises a metabolic suppressant agent.

73. The composition of any of claims 1-72, wherein the composition comprises a whole blood substitute.

74. The composition of claim 73, wherein the composition further comprises a clotting agent, a volume replacement solution, a drag reducing polymer, an anti-inflammatory agent, or a combination thereof.

75. The composition of claim 74, wherein the clotting agent comprises tranexamic acid.

76. The composition of any of claims 74-75, wherein the volume replacement solution comprises polymerized human serum albumin.

77. The composition of any of claims 74-76, wherein the drag reducing polymer comprises polyethylene glycol.

78. The composition of any of claims 74-77, wherein the anti-inflammatory agent comprises dexamethasone.

79. The composition of any of claims 74-78, wherein the composition comprises
from 25-200 mg/ml of the polymerized hemoglobin (PolyHb);
from 25-200 mg/ml of a volume replacement solution, such as polymerized human serum albumin;
from 1-50 mg/ml of a clotting agent, such as tranexamic acid;
optionally from 0.05-2 mg/ml of a drag reducing polymer, such as polyethylene glycol; and
optionally from 0.1-10 mg/ml of an anti-inflammatory agent, such as dexamethasone.

80. A method of delivering oxygen to a cell comprising exposing the cell to the HBOC composition of any of claims 1-79.

81. The method of claim 80, wherein the HBOC composition is a cell culture medium or is added to a cell culture medium.

82. The method of claim 80, further comprising infusing the HBOC composition into the cardiovascular system of a subject.

83. The method of claim 80, further comprising perfusing the tissue of a subject with the HBOC composition.

84. The method of claim 80, further comprising perfusing an organ or tissue or cells ex vivo with the HBOC composition.
